(19) United States
(12) Patent Application Publication
(10) Pub. No.: US 2019/0330220 A1
(43) Pub. Date: Oct. 31, 2019

(54) METHODS OF MAKING SUBSTITUTED PORPHYRIN PHARMACEUTICAL COMPOUNDS AND COMPOSITIONS

(71) Applicants: Duke University, Durham, NC (US); BioMimetix JV, LLC, Englewood, CO (US)

(72) Inventors: Ines Batinic-Haberle, Durham, NC (US); Artak Tovmasyan, Durham, NC (US); Zrinka Rajic Dzolic, Pozega (HR); Ivan Spasojevic, Durham, NC (US); Christopher Allen Lee, Wilbraham, MA (US)

(21) Appl. No.: 16/468,911

(22) PCT Filed: Dec. 19, 2017
(86) PCT No.: PCT/US17/67263

§ 371 Date: Jun. 12, 2019

Related U.S. Application Data
(60) Provisional application No. 62/436,743, filed on Dec. 20, 2016.

(51) Int. Cl. C07D 487/22; C07F 13/00

(52) U.S. Cl. CPC C07D 487/22; C07F 13/005

(57) ABSTRACT

Described herein are methods and intermediates useful for making substituted porphyrins, including Mn(III) orthoN-butoxyethylpyridylporphyrin, and compositions comprising the same. In some embodiments, a method of the present invention provides a composition having a certain percentage or yield (e.g., at least 80%, 85%, 90%, or 95% by weight) of a compound of the present invention.

### RELATED APPLICATION INFORMATION

This application claims the benefit of U.S. Provisional Patent Application Ser. No. 62/436,743, filed Dec. 20, 2016, the disclosure of which is incorporated herein by reference in its entirety.

### STATEMENT OF GOVERNMENT SUPPORT

This invention was made with government support under grant numbers 1 UL 1 RR024128-01 and 5-P30-CA14236-29 awarded by the National Institutes of Health. The government has certain rights in this invention.

### FIELD OF THE INVENTION

The present invention concerns methods and intermediates useful for making substituted porphyrins, including Mn(III) ortho N-butoxyethylpyridylporphyrin, along with compositions containing the same.

### BACKGROUND OF THE INVENTION

The compound Mn(III) ortho N-butoxyethylpyridylporphyrin (Formula 001; sometimes abbreviated MnTnBuOE-2-PyP⁵⁺) is known and described in Z. Rajic et al., Free Radical Biology & Medicine 53, 1828-1834 (2012).

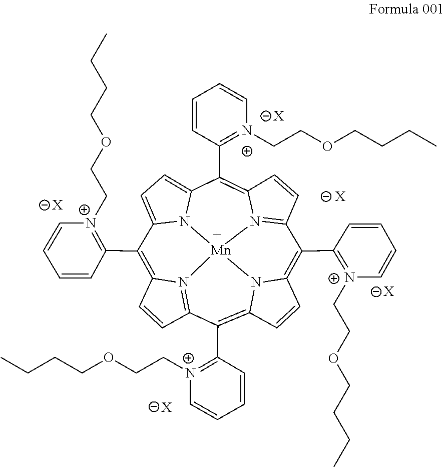

This compound is described as having a variety of activities, including, e.g., treating inflammatory lung disease, neurodegenerative conditions, radiation injury, cancer, diabetes, cardiac conditions, and sickle cell disease. See generally Batinic-Haberle et al., U.S. Pat. No. 8,616,089. This compound is, however, difficult to make in a sufficiently pure form for pharmaceutical use, and accordingly new methods of synthesis thereof would be extremely useful.

### SUMMARY OF THE INVENTION

One aspect of the present invention is directed to a method of making a compound of Formula 001:

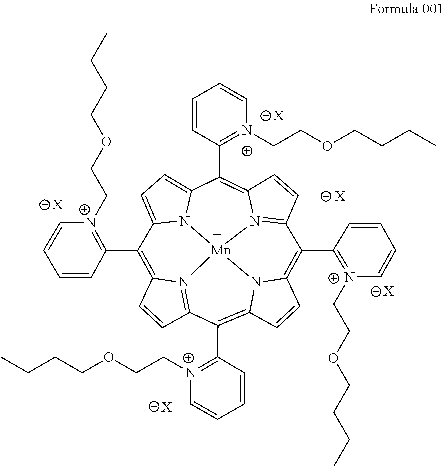

wherein X is an anion (e.g., Cl, PF₆, tosylate, besylate, mesylate, etc.), the method comprising:

(a) providing a compound of Formula 001-2:

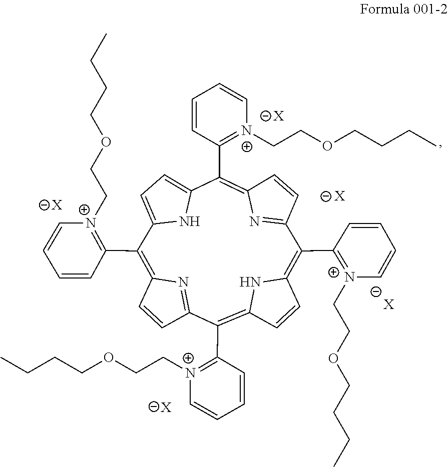

wherein X is an anion (e.g., Cl, PF₆, tosylate, besylate, mesylate, etc.) in an aqueous solution at a pH of from 10 to 12 (e.g., 11), then

(b) combining MnCl₂×4 H₂O into said aqueous solution to produce a mixed solution; and then

(c) oxygenating said mixed solution while concurrently

(d) monitoring and periodically adjusting the pH of said mixed solution to maintain a pH thereof between 7.6 or 7.8 and 8.2 or 8.4 (e.g., to maintain a pH of 8), while continuing to oxygenate said mixed solution for a time sufficient to produce said compound of Formula 001.

Another aspect of the present invention is directed to a method of making a compound of Formula 001-2

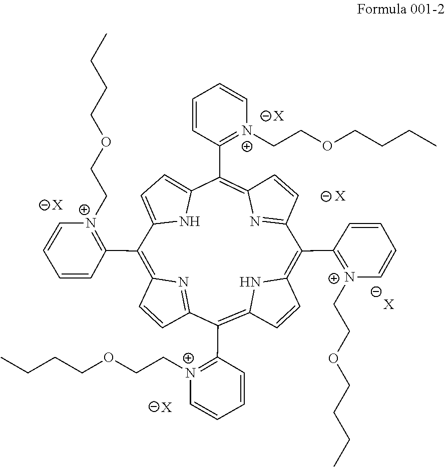

wherein X is an anion (e.g., Cl, PF₆, tosylate, besylate, mesylate, etc.), the method comprising the steps of:

(a) providing compound H₂T-2-PyP in a heated solution of a polar aprotic solvent (e.g., dimethylformamide) with tri-n-octylamine (Oct₃N)

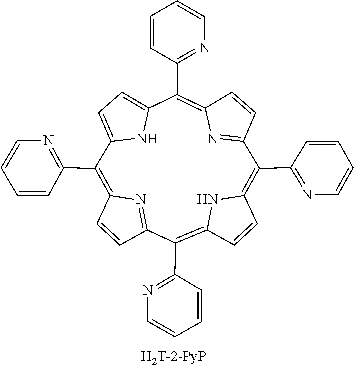 [SMILES: C1=Cc2nc1c(-c1ccccn1)c1ccc([nH]1)c(-c1ccccn1)c1nc(c(-c3ccccn3)c3ccc([nH]3)c2-c2ccccn2)C=C1]

wherein said heated solution is purged of oxygen (e.g., by sparging with an inert gas such as nitrogen or argon); then

(b) combining said heated solution with 2-butoxyethyl p-toluenesulfonate to produce a liquid mixture;

(c) maintaining said liquid mixture at an elevated temperature (e.g., 85 to 105° C.) for a time (e.g., 45-60 hours) sufficient to produce an intermediate product (i.e., BMX-001-2-OTs) in an intermediate liquid; then

(d) optionally combining said intermediate liquid with a flocculant (e.g. an organic or inorganic flocculant, such as powdered cellulose (e.g., Solka floc)) so that the intermediate product partitions with the flocculant;

(e) separating said flocculant when present from said intermediate liquid (e.g., by filtration, settling, centrifugation, or a combination thereof), then

(f) washing said flocculant with an aqueous wash solution to produce an aqueous solution carrying said intermediate reaction product; and

(g) combining said aqueous solution with a salt of said anion to produce said compound of Formula 001-2.

Another aspect of the present invention is directed to a method of making a compound of Formula 002:

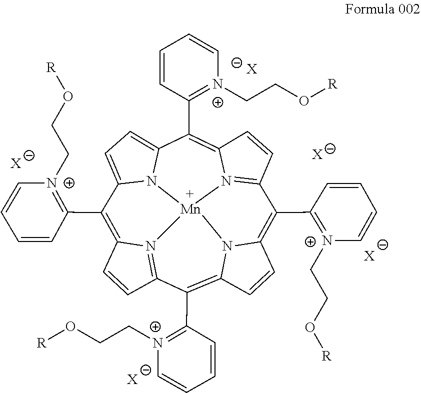

wherein each R is independently a C4-C12 alkyl and X is an anion (e.g., Cl, PF₆, tosylate, besylate, mesylate, etc.), the method comprising:

(a) providing a compound of Formula 002-2:

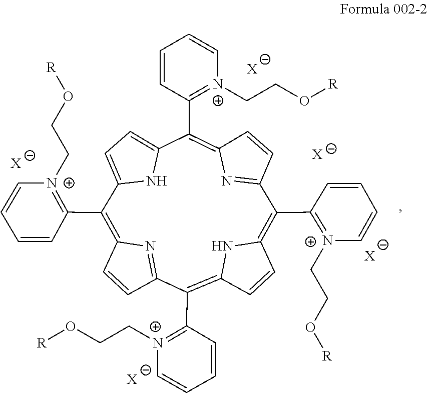

wherein each R is independently a C4-C12 alkyl and X is an anion (e.g., CI, PF₆, tosylate, besylate, mesylate, etc.) in an aqueous solution at a pH of from 10 to 12 (e.g., 11), then

(b) combining MnCl₂×4 H₂O into said aqueous solution to produce a mixed solution; and then

(c) oxygenating said mixed solution while concurrently

(d) monitoring and periodically adjusting the pH of said mixed solution to maintain a pH thereof between 7.6 or 7.8 and 8.2 or 8.4 (e.g., to maintain a pH of 8), while continuing to oxygenate said mixed solution for a time sufficient to produce said compound of Formula 002.

Another aspect of the present invention is directed to a method of making a compound of Formula 002-2

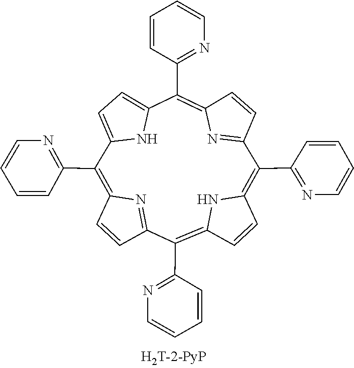

wherein each R is independently a C4-C12 alkyl and X is an anion (e.g., Cl, PF₆, tosylate, besylate, mesylate, etc.), the method comprising the steps of:

(a) providing compound H₂T-2-PyP in a heated solution of a polar aprotic solvent (e.g., dimethylformamide) with tri-n-octylamine (Oct₃N)

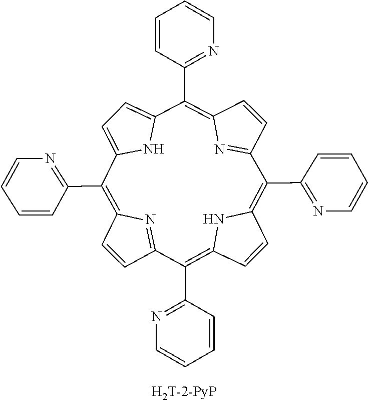 [SMILES: C1=Cc2nc1c(-c1ccccn1)c1ccc([nH]1)c(-c1ccccn1)c1nc(c(-c3ccccn3)c3ccc([nH]3)c2-c2ccccn2)C=C1]

wherein said heated solution is purged of oxygen (e.g., by sparging with an inert gas such as nitrogen or argon); then

(b) combining said heated solution with 2-alkoxyethyl p-toluenesulfonate to produce a liquid mixture;

(c) maintaining said liquid mixture at an elevated temperature (e.g., 85 to 105° C.) for a time (e.g., 45-60 hours) sufficient to produce an intermediate product (i.e., BMX-001-2-OTs) in an intermediate liquid; then

(d) optionally combining said intermediate liquid with a flocculant (e.g. an organic or inorganic flocculant, such as powdered cellulose (e.g., Solka floc)) so that the intermediate product partitions with the flocculant;

(e) separating said flocculant when present from said intermediate liquid (e.g., by filtration, settling, centrifugation, or a combination thereof), then

(f) washing said flocculant with an aqueous wash solution to produce an aqueous solution carrying said intermediate reaction product; and

(g) combining said aqueous solution with a salt of said anion to produce said compound of Formula 002-2.

A further aspect of the present invention is directed to a pharmaceutical composition comprising metallated pyridyl-porphyrins in a pharmaceutically acceptable carrier, wherein at least 80, 85, 90 or 95 percent by weight of all of said metallated pyridyl-porphyrins in said composition is a compound of Formula 001 or Formula 002

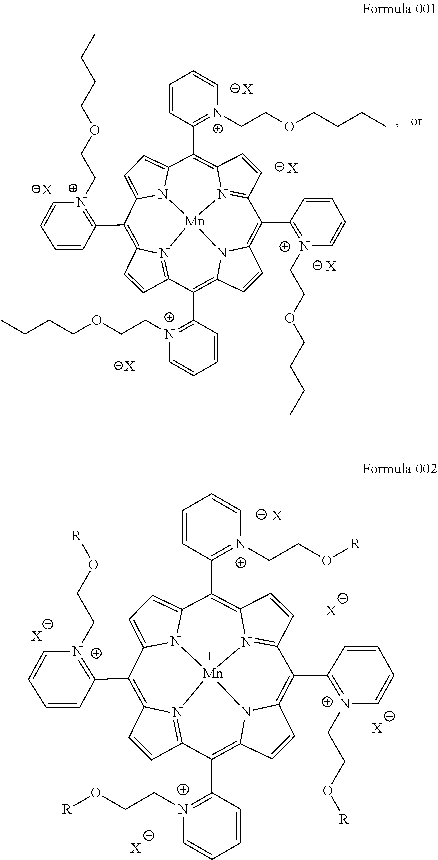

wherein X is a pharmaceutically acceptable anion and each R is independently a C4-C12 alkyl.

Another aspect of the present invention is directed to use of a composition of the present invention in treating inflammatory lung disease, neurodegenerative disease, radiation injury, cancer, diabetes, cardiac conditions, and/or sickle cell disease.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 shows the structures of Mn(III) porphyrins.

FIG. 2 shows a TLC plate and ESI-MS analyses of both the crude mixture and the materials recovered from TLC spots of the “MeOBu/3-Py” system, which was initially thought to yield MnTMOBu-3-PyP⁵⁺. TLC-SiO₂ was carried out in 1:1:8=saturated KNO₃ H₂O:H₂O:CH₃CN system. ESI-MS peaks in the m/z 370-500 region correspond to ion-pairs (MnP⁵⁺+2HFBA⁻)³⁺/3.

FIG. 3 shows the distribution of the mixture of species bearing “n” methoxyalkyl groups and “4-n” methyl groups (n=0 to 4) on pyridyl nitrogens in different N-methoxyalkylpyridylporphyrin preparations.

FIG. 4 shows the levels of overall methylation (as opposed to methoxyalkylation) in different N-methoxyalkylpyridylporphyrins preparations.

FIG. 5 shows the proposed reaction mechanisms for the competing alkoxyalkylation and methylation reactions of N-pyridylporphyrins in the presence of alkoxyalkyl tosylates. Pyridine has been used as a surrogate species for the pyridyl moieties of the N-pyridylporphyrins. R=methyl, and n=0, 2, 3, or 4 for methoxyethyl, methoxybutyl, methoxypentyl or methoxyhexyl tosylates, respectively. R=butyl and n=0 for butoxyethyl tosylate case.

FIG. 6 shows the Gibbs free energy profile calculated at M06-2X/6-31 l++G(2d,p)//M06-2X/6-31+G(d) DFT level for the species associated with the mechanisms given in FIG. 5. Compression and ionic pair effects were taken into account where appropriate.

FIG. 7 shows a comparison of the Gibbs free energy for the MeOEtOTs and nBuOEtOTs systems calculated at M06-2X/6-311++G(2d,p)//M06-2X/6-31+G(d) DFT level for the species associated with the mechanisms given in FIG. 5. Compression and ionic pair effects were taken into account where appropriate.

### DETAILED DESCRIPTION OF ILLUSTRATIVE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

“Pharmaceutically acceptable” as used herein means that the compound, anion, or composition is suitable for administration to a subject to achieve the treatments described herein, without unduly deleterious side effects in light of the severity of the disease and necessity of the treatment.

Provided according to embodiments of the present invention are methods of making a compound of Formula 002:

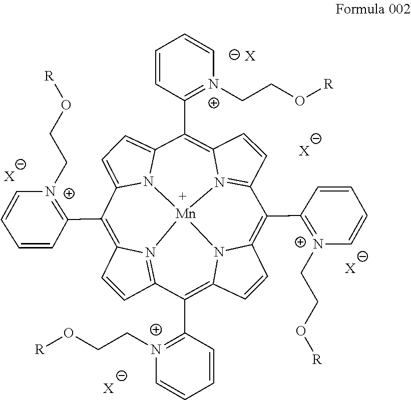

wherein each R is independently a C4-C12 alkyl and X is an anion (e.g., Cl, PF₆, tosylate, besylate, mesylate, etc.). In some embodiments, all R groups in a compound of Formula 002 are the same and are a C4-C12 alkyl (e.g., a C4, C5, C6, C7, C8, C9, C10, Cl 1, or C12 alkyl). In some embodiments, R is a C4-C6 alkyl. In some embodiments, provided is a method of making a compound of Formula 001:

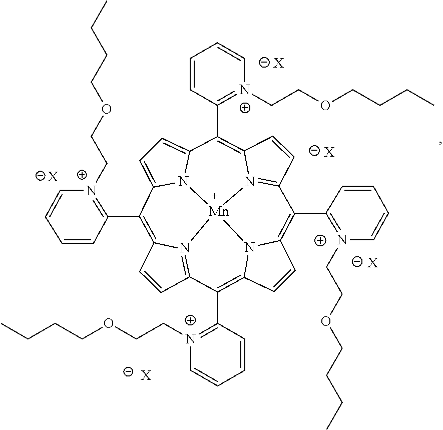

wherein X is an anion (e.g., CI, PF₆, tosylate, besylate, mesylate, etc.).

In some embodiments, a method of the present invention comprises (a) providing a compound of Formula 002-2:

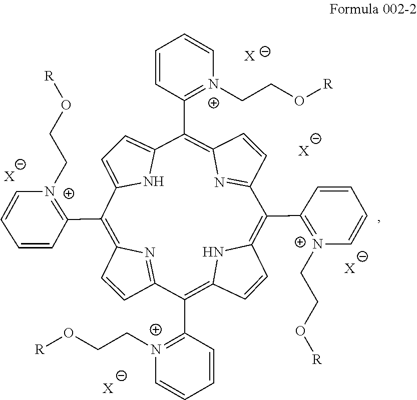

wherein each R is independently a C4-C12 alkyl and X is an anion (e.g., Cl, PF₆, tosylate, besylate, mesylate, etc.), in an aqueous solution at a pH from 10 to 12, (b) combining MnCl₂.4H₂O into the aqueous solution to produce a mixed solution; (c) oxygenating the mixed solution; and (d) monitoring and periodically adjusting the pH of the mixed solution to maintain a pH thereof from 7.6 or 7.8 to 8.2 or 8.4 (e.g., to maintain a pH of 8), while continuing to oxygenate the mixed solution for a time sufficient to produce the compound of Formula 002. The pH may be monitored continuously, regularly (e.g., every 10, 20, 30, or 40 minutes), and/or discontinuously while oxygenating the mixed solution. In some embodiments, all R groups in a compound of Formula 002-2 are the same and are a C4-C12 alkyl (e.g., a C4, C5, C6, C7, C8, C9, C10, C11, or C12 alkyl). In some embodiments, R is a C4-C6 alkyl.

In some embodiments, a method of the present invention comprises (a) providing a compound of Formula 001-2:

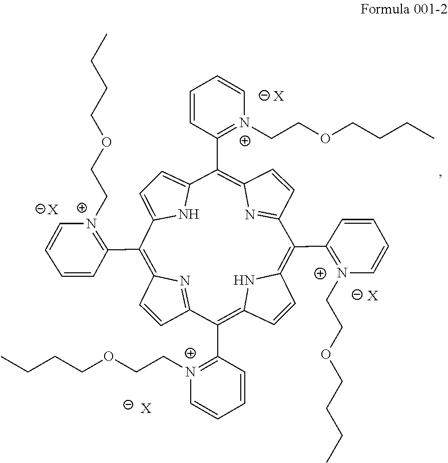

wherein X is an anion (e.g., Cl, PF₆, tosylate, besylate, mesylate, etc.), in an aqueous solution at a pH from 10 to 12, (b) combining MnCl₂.4H₂O into the aqueous solution to produce a mixed solution; (c) oxygenating the mixed solution; and (d) monitoring and periodically adjusting the pH of the mixed solution to maintain a pH thereof from 7.6 or 7.8 to 8.2 or 8.4 (e.g., to maintain a pH of 8), while continuing to oxygenate the mixed solution for a time sufficient to produce the compound of Formula 001.

In a method of the present invention, the aqueous solution may have a pH of 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, or 12.0. In some embodiments, the aqueous solution may have a pH in a range from 10.5 to 11.5. In some embodiments, the aqueous solution may have a pH of about 11.

While oxygenating the mixed solution, the mixed solution may be maintained at and/or adjusted to a pH of 7.6, 7.7, 7.8, 7.9, 8.0, 8.1, 8.2, 8.3, or 8.4. In some embodiments, while oxygenating the mixed solution, the mixed solution may be maintained at and/or adjusted to a pH of about 8.0, or the pH may be in a range of or between a pH of 7.6 or 7.8 and 8.2 or 8.4. During the monitoring step, if the mixed solution has a pH of less than 7.6 or 7.8, then the pH of the mixed solution may be adjusted by adding a base to the mixed solution. Alternatively, if during the monitoring step the mixed solution has a pH of greater than 8.2 or 8.4, then the pH of the mixed solution may be adjusted by adding an acid to the mixed solution. The monitoring step may carried out by contacting the mixed solution during the oxygenating step with a pH sensor and/or detector.

The step of providing the compound of Formula 001-2 or Formula 002-2 may be carried out by providing a composition of pyridyl porphyrins that comprises the compound of Formula 001-2 or Formula 002-2, respectively, along with one or more different pyridyl porphyrins. The composition of pyridyl porphyrins may comprise the compound of Formula 001-2 or Formula 002-2 in an amount of at least about 80, 81, 82, 83, 84, 85, 86, 87, 88, 89, 90, 91, 92, 93, 94, 95, 96, 97, 98, or 99 percent or more by weight of all pyridyl porphyrins.

A method of the present invention may produce the compound of Formula 002 in an amount of at least about 80, 81, 82, 83, 84, 85, 86, 87, 88, 89, 90, 91, 92, 93, 94, 95, 96, 97, 98, or 99 percent or more by weight of all manganese pyridyl-porphyrins produced from the compound of Formula 002-2 or the composition comprising the compound of Formula 002-2.

In some embodiments, a method of the present invention may produce the compound of Formula 001 in an amount of at least about 80, 81, 82, 83, 84, 85, 86, 87, 88, 89, 90, 91, 92, 93, 94, 95, 96, 97, 98, or 99 percent or more by weight of all manganese pyridyl-porphyrins produced from the compound of Formula 001-2 or the composition comprising the compound of Formula 001-2.

In some embodiments, not more than 20, 19, 18, 17, 16, 15, 14, 13, 12, 11, 10, 9, 8, 7, 6, or 5 percent or less by weight of all manganese pyridyl-porphyrins produced from a method of the present invention consists of compounds of Formulas (iii), (iv), (v), (vi), (vii) and (viii):

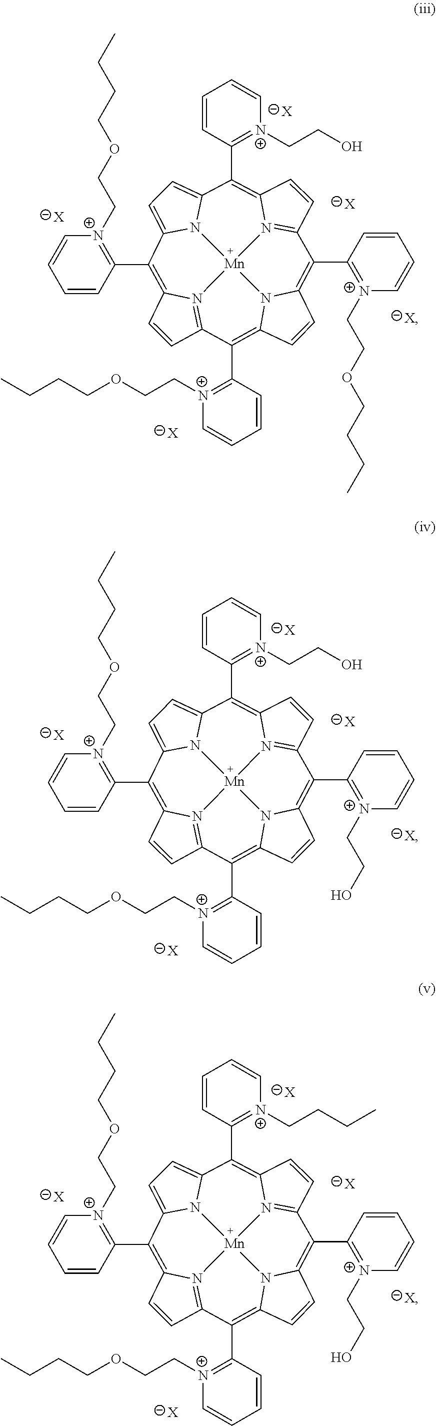

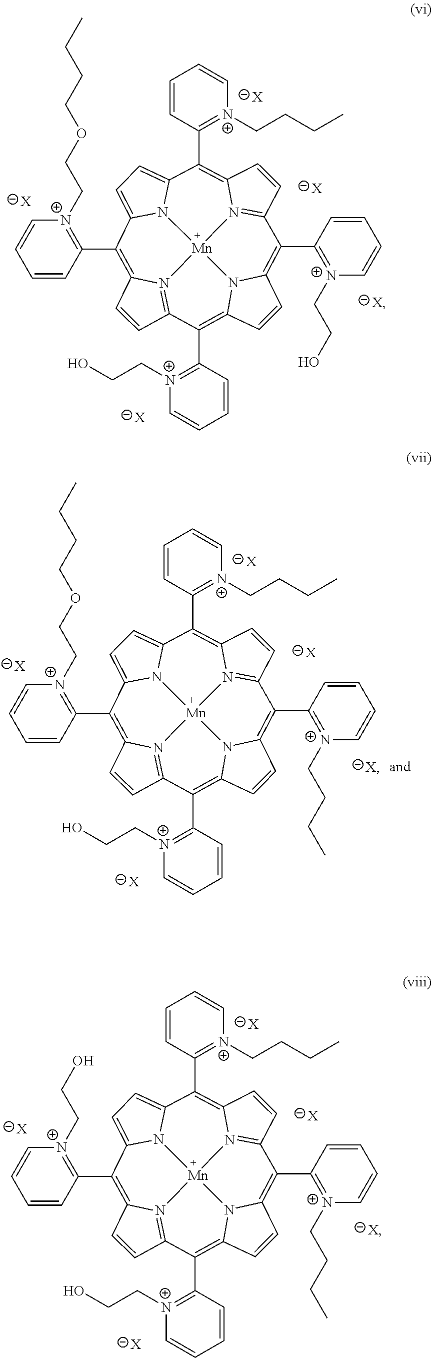

wherein X is an anion as described above.

In some embodiments, not more than 20, 19, 18, 17, 16, 15, 14, 13, 12, 11, 10, 9, 8, 7, 6, or 5 percent or less by weight of all manganese pyridyl-porphyrins produced from a method of the present invention consists of compounds of Formulas (iiia), (iva), (va), (via), (viia) and (viiia):

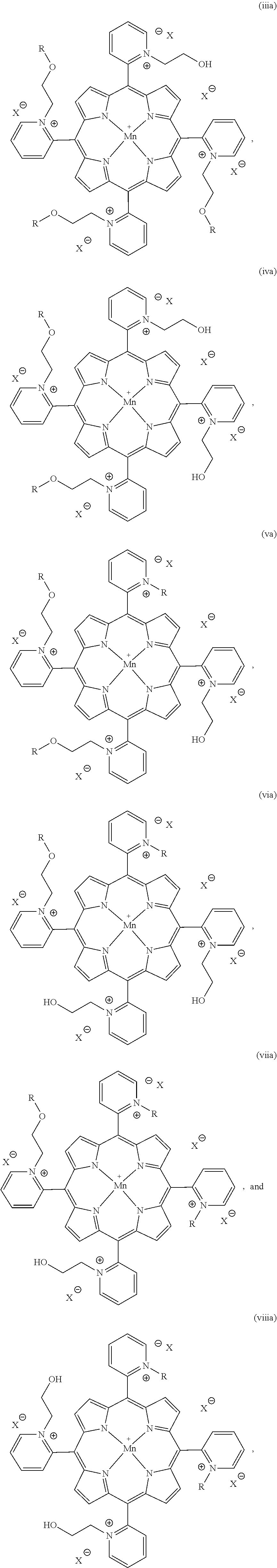

wherein each R is independently a C4-C12 alkyl and X is an anion as described above. In some embodiments, all R groups in a compound of Formula (iiia), (iva), (va), (via), (viia) or (viiia) are the same and are a C4-C12 alkyl (e.g., a C4, C5, C6, C7, C8, C9, C10, C11, or C12 alkyl). In some embodiments, R is a C4-C6 alkyl.

According to some embodiments, provided is a method of making a compound of Formula 002-2:

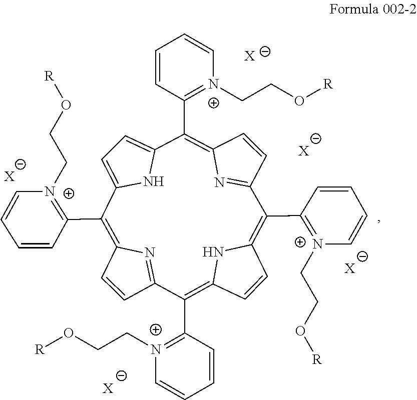

wherein each R is independently a C4-C12 alkyl and X is an anion (e.g., CI, PF₆). In some embodiments, all R groups in a compound of Formula 002-2 are the same and are a C4-C12 alkyl (e.g., a C4, C5, C6, C7, C8, C9, C10, C11, or C12 alkyl). In some embodiments, R is a C4-C6 alkyl. In some embodiments, provided is a method of making a compound of Formula 001-2

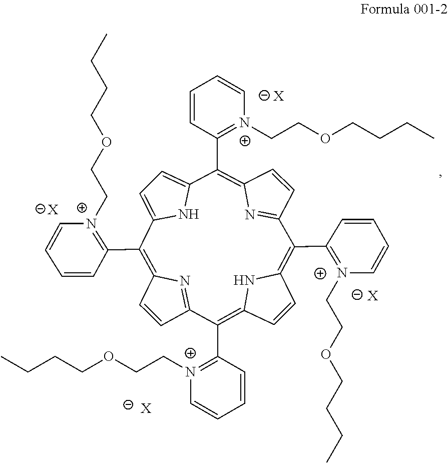

wherein X is an anion (e.g., CI, PF₆). A method of making a compound of Formula 002-2 or Formula 001-2 may comprise the steps of: (a) providing compound H₂T-2-PyP in a heated solution of a polar aprotic solvent (e.g., dimethylformamide) with tri-n-octylamine (Oct₃N), tri-isopropanolamine, tri-n-decylmaine and/or tri-n-dodecylamine

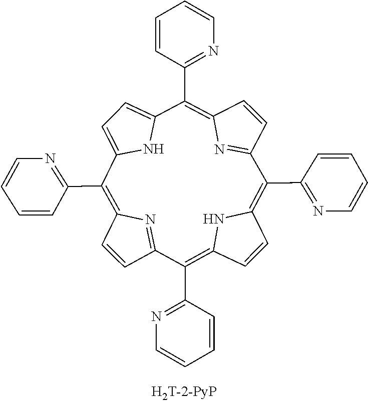 [SMILES: C1=Cc2nc1c(-c1ccccn1)c1ccc([nH]1)c(-c1ccccn1)c1nc(c(-c3ccccn3)c3ccc([nH]3)c2-c2ccccn2)C=C1]

wherein the heated solution is purged of oxygen; (b) combining the heated solution with 2-alkoxyethyl p-toluenesulfonate (e.g., 2-butoxyethyl p-toluenesulfonate for a compound of Formula 001-2) to produce a liquid mixture; (c) maintaining the liquid mixture at an elevated temperature for a time sufficient to produce an intermediate product in an intermediate liquid; (d) optionally combining the intermediate liquid with a flocculant so that the intermediate product partitions with the flocculant; (e) separating the flocculant, when present, from the intermediate liquid; (f) washing the flocculant with an aqueous wash solution to produce an aqueous solution carrying the intermediate reaction product; and (g) combining the aqueous solution with a salt of the anion to produce the compound of Formula 002-2 or Formula 001-2.

In some embodiments, the heated solution may be purged of oxygen by sparging with an inert gas such as nitrogen or argon.

Some embodiments include maintaining the liquid mixture at an elevated temperature in a range of about 85 to about 105° C. for a time in a range of about 45 to about 60 hours sufficient to produce an intermediate product in an intermediate liquid. In some embodiments, the intermediate product is BMX-001-2-OTs. In some embodiments, the liquid mixture may be maintained at an elevated temperature of about 85, 90, 95, 100, or 105° C., or any range therein, for a time of about 45, 50, 55, or 60 hours, or any range therein.

The flocculant may be an organic or inorganic flocculant, such as, e.g., powdered cellulose (e.g., Solka floc). The flocculant may be separated from the intermediate liquid using any suitable method, such as, e.g., by filtration, settling, centrifugation, or a combination thereof.

In some embodiments, the combining step (b) is carried out with a 2-alkoxyethyl p-toluenesulfonate (e.g., a 2-butoxyethyl p-toluenesulfonate composition) comprising less than 1 weight percent (relative to said 2-alkoxyethyl p-toluenesulfonate) of tetrahydrofuran (THF). While not wishing to be bound to any particular theory, this step may serve to remove tetrahydrofurane from 2-alkoxyethyl p-toluenesulfonate (e.g., 2-butoxyethyl p-toluenesulfonate) and/or serve to reduce undesirable products other than BMX-001 in the final composition.

Tri-n-octylamine, tri-isopropanolamine, tri-n-decylmaine and/or tri-n-dodecylamine may be present in the polar aprotic solvent in an amount of about 5 to about 25 molar excess over H2T-2-PyP. For example, tri-n-octylamine, tri-isopropanolamine, tri-n-decylmaine and/or tri-n-dodecylamine may be present in the polar aprotic solvent in an amount of about 5, 10, 15, or 20 molar excess compared to H2T-2-PyP.

A method of the present invention may produce one or more contaminating intermediate compound(s). Contaminating intermediate compounds due to erroneous substitution on the pyridyl nitrogen may include a compound of Formula (ia) and/or (iia):

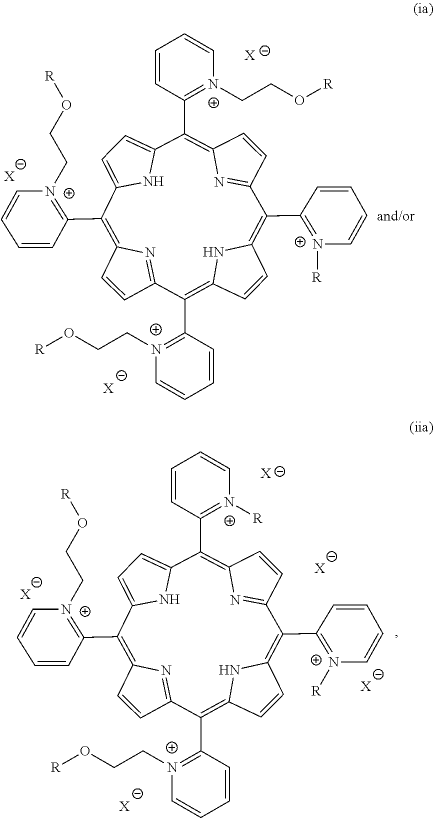

wherein each R is independently a C4-C12 alkyl and X is an anion (e.g., Cl, PF₆, tosylate, besylate, mesylate, etc.). In some embodiments, all R groups in a compound of Formula (ia) or (iia) are the same and are a C4-C12 alkyl (e.g., a C4, C5, C6, C7, C8, C9, C10, C11, or C12 alkyl). In some embodiments, R is a C4-C6 alkyl.

In some embodiments, contaminating intermediate compounds due to erroneous substitution on the pyridyl nitrogen may include a compound of Formula (i) and/or (ii):

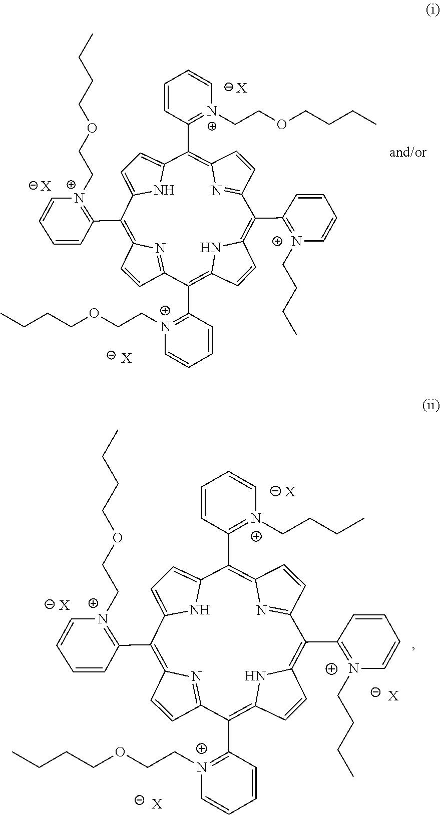

wherein X is an anion (e.g., Cl, PF₆, tosylate, besylate, mesylate, etc.).

Contaminating metallated compounds due to cleavage of the alkoxyethyl (e.g., butoxyethyl) chain during metallation (taking into consideration contaminants as described above, that may already be present from the prior step), may include:

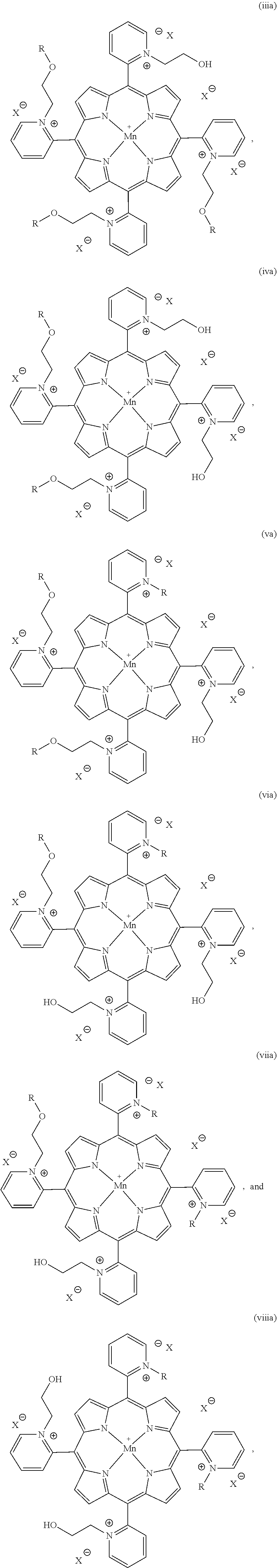

wherein each R is independently a C4-C12 alkyl and X is an anion (e.g., Cl, PF₆, tosylate, besylate, mesylate, etc.).

In some embodiments, contaminating metallated compounds due to cleavage of the butoxyethyl chain during metallation (taking into consideration contaminants as described above, that may already be present from the prior step), may include:

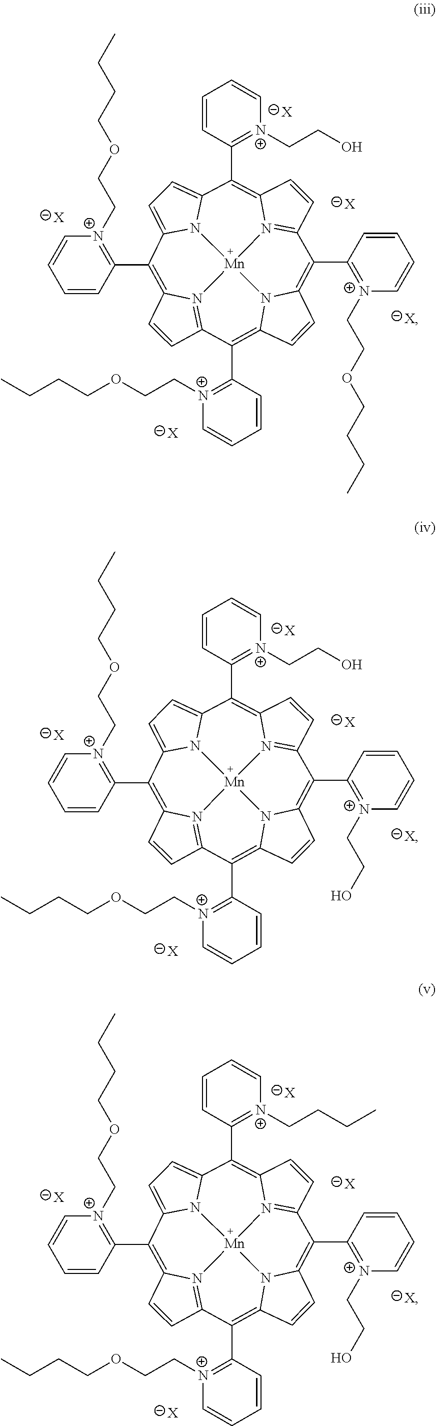

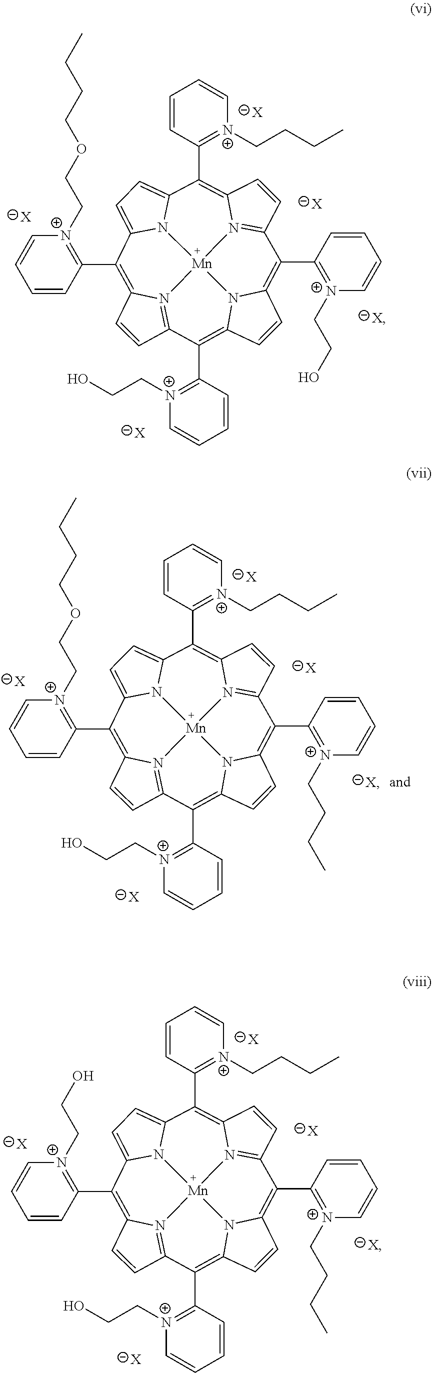

wherein X is an anion (e.g., CI, PF₆, tosylate, besylate, mesylate, etc.).

Compounds and compositions of the present invention may be used for treating any of a variety of conditions in human and other mammalian subjects, including but not limited to treating inflammatory lung disease, neurodegenerative disease, radiation injury, cancer, diabetes, cardiac conditions, sickle cell disease, etc. See generally Batinic-Haberle et al., U.S. Pat. No. 8,616,089.

In some embodiments, a pharmaceutical composition is provided comprising a compound prepared according to a method of the present invention. In some embodiments, a pharmaceutical composition may comprise metallated pyridyl-porphyrins in a pharmaceutically acceptable carrier, wherein at least about 80, 81, 82, 83, 84, 85, 86, 87, 88, 89, 90, 91, 92, 93, 94, 95, 96, 97, 98, 99, or 100 percent by weight of all of said metallated pyridyl-porphyrins in said composition is a compound of Formula 001 or a compound of Formula 002:

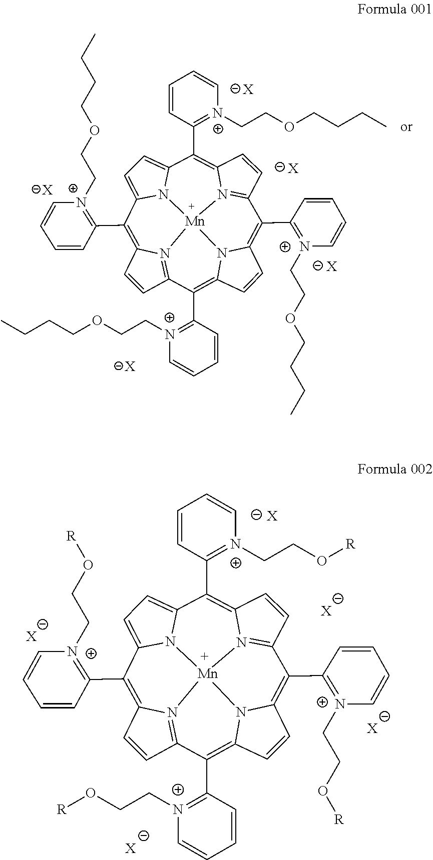

wherein X is a pharmaceutically acceptable anion and each R is independently a C4-C12 alkyl.

The pharmaceutically acceptable anion X may be selected from the group consisting of Cl, PF₆ tosylate, mesylate, and besylate. The pharmaceutically acceptable carrier may be an aqueous carrier.

A pharmaceutical composition of the present invention may comprise, excluding the weight of the pharmaceutically acceptable carrier in the composition, less than about 2, 1.8, 1.5, 1.3, or 1 percent by weight free manganese.

In some embodiments, not more than 20, 19, 18, 17, 16, 15, 14, 13, 12, 11, 10, 9, 8, 7, 6, 5, 4, 3, 2, or 1 percent by weight of all metallated pyridyl-porphyrins in the composition consist of compounds of Formulas (iiia), (iva), (va), (via), (viia) and (viiia):

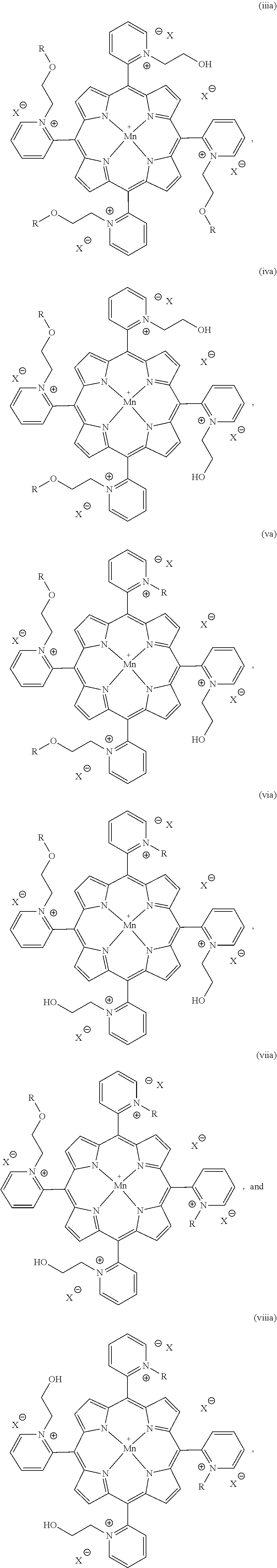

wherein each R is independently a C4-C12 alkyl and X is an anion (e.g., Cl, PF₆, tosylate, besylate, mesylate, etc.).

In some embodiments, not more than 20, 19, 18, 17, 16, 15, 14, 13, 12, 11, 10, 9, 8, 7, 6, 5, 4, 3, 2, or 1 percent by weight of all metallated pyridyl-porphyrins in the composition consist of compounds of Formulas (iii), (iv), (v), (vi), (vii) and (viii):

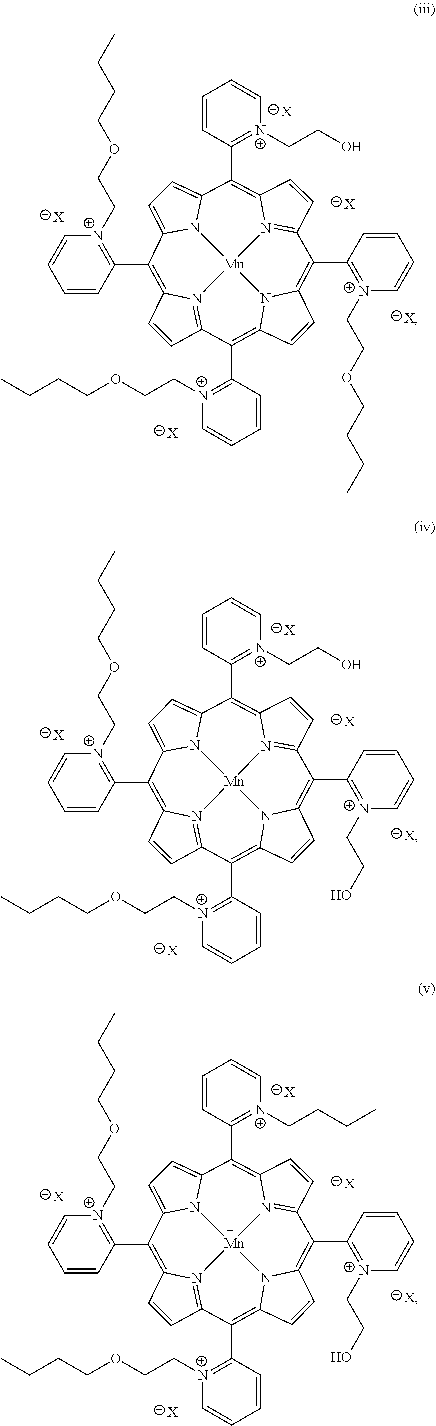

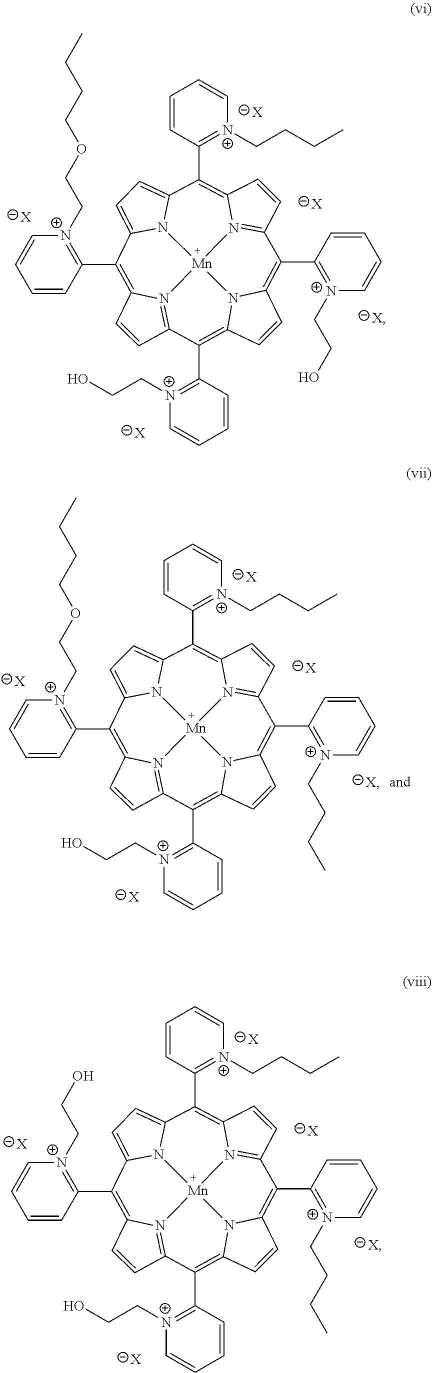

where X is an anion as given above.

In some embodiments, a compound of the present invention may have one or more (e.g., 1, 2, 3, 4, or more) atropisomers. Thus, a composition of the present invention may comprise one or more (e.g., 1, 2, 3, 4, or more) atropisomers of a compound, such as, for example, a compound of Formula 001 or a compound of Formula 002.

In some embodiments, a compound of Formula 001 or a compound of Formula 002 may have four atropisomers for which the structure of the 4 atropisomers is identical except for the position of the side chain (e.g., —CH₂—CH₂—O—CH₂—CH₂—CH₂—CH₃) on the each of the four pyridyl groups. The atropisomers may be created by the fact that they must extend either above or below the plane of the porphyrin ring and they may be held in place by steric hindrance and do not readily interconvert. The four atropisomers may be as follows: Atropisomer #1—all four side chains on the same side of the porphyrin ring (i.e., alpha-alpha-alpha-alpha), Atropisomer #2—three side chains on one side of the porphyrin ring and one on the other side (i.e., alpha-alpha-alpha-beta), Atropisomer #3—two chains above the ring and two below with them alternating position (i.e., alpha-beta-alpha-beta), and Atropisomer #4—two chains above the ring and two below the ring with the side chains adjacent to each other (i.e., alpha-alpha-beta-beta). In some embodiments, a compound of Formula 001 may have a structure represented by:

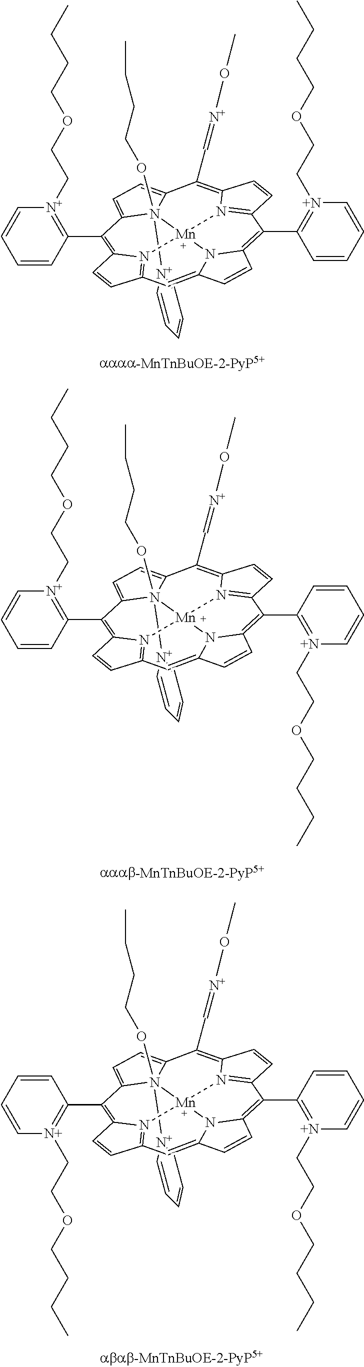

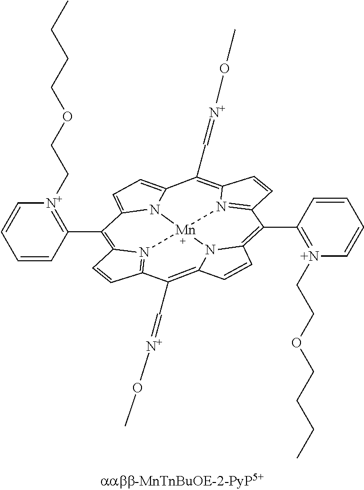

In some embodiments, a compound of Formula 001 of the present invention may have and/or a composition of the present invention may comprise Atropisomer #1 (i.e., alpha-alpha-alpha-alpha) in an amount of about 5% to about 15% by weight of the compound of Formula 001, Atropisomer #2 (i.e., alpha-alpha-alpha-beta) in an amount of about 45% to about 55% by weight of the compound of Formula 001, Atropisomer #3 (i.e., alpha-beta-alpha-beta) in an amount of about 10% to about 20% by weight of the compound of Formula 001, and Atropisomer #4 (i.e., alpha-alpha-beta-beta) in an amount of about 20% to about 30% by weight of the compound of Formula 001.

A pharmaceutical composition of the present invention may be used in treating inflammatory lung disease, neurodegenerative disease, radiation injury, cancer, diabetes, cardiac conditions, and/or sickle cell disease in a subject. “Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the disease or disorder and/or there is a delay in the progression of the disease or disorder.

In some embodiments, a pharmaceutical composition of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a pharmaceutical composition of the present invention.

The present invention finds use in both veterinary and medical applications. Subjects suitable to be treated with a pharmaceutical composition of the invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects.

In some embodiments, a pharmaceutical composition of the present invention may be carried out on animal subjects, particularly mammalian subjects such as mice, rats, dogs, cats, livestock and horses for veterinary purposes and/or for drug screening and/or drug development purposes.

A compound of the present invention may be formulated for administration in a pharmaceutical carrier in accordance with known techniques. See, e.g., Remington, The Science And Practice of Pharmacy (9th Ed. 1995). In the manufacture of a pharmaceutical formulation according to the invention, the compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the compound. One or more compounds may be incorporated in the formulations of the invention, which may be prepared by any of the well-known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular compound which is being used.

Formulations suitable for oral administration may be presented in discrete units, such as capsules, cachets, lozenges, or tablets, each containing a predetermined amount of the compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the compound and a suitable carrier (which may contain one or more accessory ingredients as noted above). In general, the formulations of the invention are prepared by uniformly and intimately admixing the compound with a liquid or finely divided solid carrier, or both, and then, if necessary, shaping the resulting mixture. For example, a tablet may be prepared by compressing or molding a powder or granules containing the compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the compound with one or more conventional solid carriers, for example, cocoa butter, and then shaping the resulting mixture. Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. Of course, the liposomal formulations containing the compounds disclosed herein or salts thereof may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidylcholines, and lecithin.

In addition to compound(s) of the present invention, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multi-dose use. Of course, as indicated, the pharmaceutical compositions of the present invention may be lyophilized using techniques well known in the art.

As noted above, the present invention provides pharmaceutical compositions comprising a compound of the present invention (including the pharmaceutically acceptable salts thereof), in pharmaceutically acceptable carriers for oral, rectal, topical, buccal, parenteral, intramuscular, intradermal, or intravenous, and transdermal administration.

The effective amount (e.g., therapeutically effective or treatment effective amount) or dosage of any specific compound as described herein, for use in any specific method as described herein, will vary depending on factors such as the condition being treated, the route of administration, the general condition of the subject (e.g., age, gender, weight, etc.), etc. In general (e.g., for oral or parenteral administration), the dosage may be from about 0.01, 0.05, or 0.1 milligram per kilogram subject body weight (mg/kg), up to about 1, 5, or 10 mg/kg. For topical administration, the compound may be included in a pharmaceutically acceptable composition to be applied in any suitable amount, typically from 0.01, 0.1, or 1 percent by weight, up to 10, 20, or 40 percent by weight, or more, of the weight of the composition, again depending on factors such as the condition being treated, condition of the subject, etc.

The compounds described herein may be administered directly or through the administration to the subject of a pharmaceutically acceptable prodrug which is in turn converted to the active agent in vivo. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. Nos. 6,680,324 and 6,680,322.

The present invention is explained in greater detail in the following non-limiting Examples.

### Examples 1-8

### Synthesis of BMX-001 from 2-Butoxyethanol and 5,10,15,20-tetrakis(2-pyridyl)porphyrin (H₂T-2-PyP)

These examples describe the synthesis of the above compound from the above starting reagents by the following overall scheme:

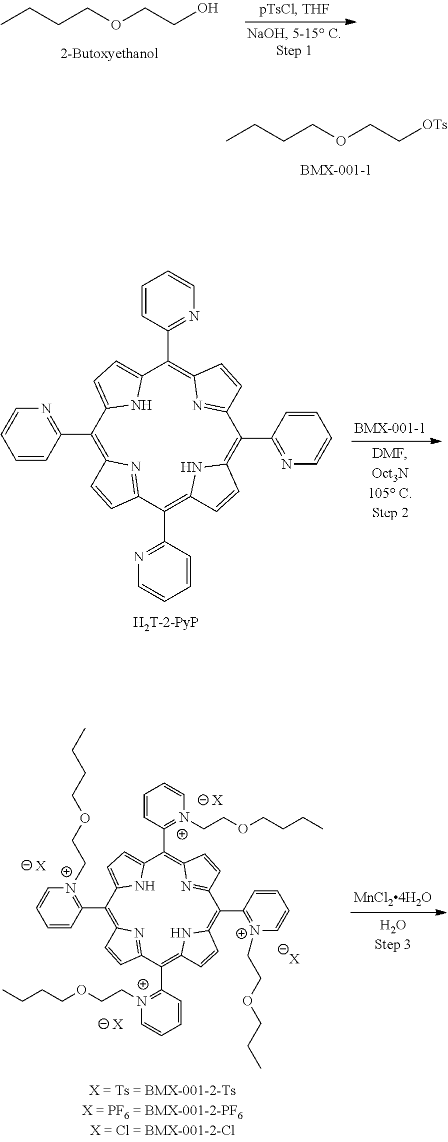

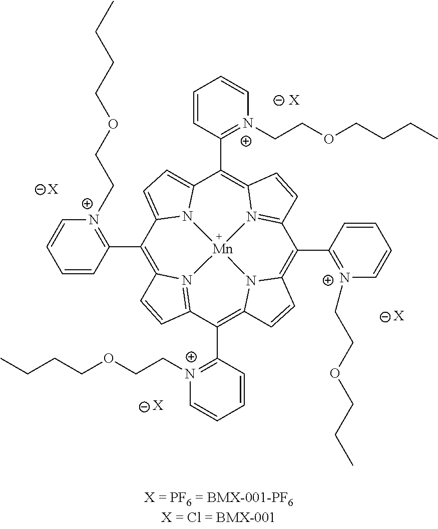

H₂T-2-PyP is known. See, e.g., I. Batinic-Haberle et al., Dalton Trans. 2004, 1696-1702. BMX-001-1, or 2-butoxyethyl p-toluenesulfonate, are also known. See, e.g., R. Tipson, On esters of p-toluenesulfonic acid, J. Org. Chem. 9, 235-241 (1944) “Ts” above refers to p-toluenesulfonate. The steps of the foregoing overall scheme and particular embodiments thereof are explained in greater detail below.

### Example 1

### Synthesis of BMX-001-1

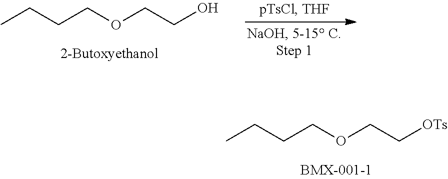

2-Butoxyethanol (7.0 kg, 59.2 mol) and water (12 L, House RO water) were charged to a 100 L Slytherm glass jacketed reactor equipped with a mechanical stirrer, thermocouple probe, and distillation head. The batch was stirred and cooled to 0-5° C. under static nitrogen. A solution of 50 wt % NaOH (5.45 kg, 68.1 mol) was added while maintaining 0-30° C. Note: the addition required 30 minutes to complete. A solution of p-toluenesulfonyl chloride (10.2 kg, 53.3 mol) in tetrahydrofuran (THF) (28.0 L) was added to the batch while maintaining 5-20° C. Note: The addition required 90 minutes to complete. The batch was warmed to 20-25° C. and stirred for 1 hour. After 1 hour, the organic layer was sampled, concentrated, and analyzed by ¹H NMR (CDCl3) for residual p-toluenesulfonyl chloride. After 1 hour at 20-25° C., the p-toluenesulfonyl chloride content was <1 wt % and the reaction was deemed complete. MTBE (21 L) was added and the batch adjusted to pH=7.0-7.5 by adding aqueous 6 M HCl (1.7 L). Note: the initial pH was 14 and the final pH was 7.0. The organic layer was separated, washed with a solution of aqueous saturated Brine (1.4 L) in water (12.6 L, House RO water), and concentrated by vacuum distillation (23-26 inches of Hg, 40-45° C. batch temp) until distillation ceased. The batch was cooled to 20-30° C. and washed with water (4×28 L, House RO water). MTBE (14 L) was added and the batch was washed with a solution of aqueous saturated Brine (1.4 L) in water (12.6 L, House RO water). The organic layer was then diluted with THF (14.0 L and the batch was concentrated by vacuum distillation (23-26 inches of Hg, 40-45° C. batch temp) until distillation ceased. The batch was then cooled to 20-25° C. and assayed for residual water (Karl Fisher <0.1 wt %) and THF (¹H NMR (CDCl₃) 8 wt % THF). After passing the residual water specification of <0.1 wt %, the batch was polish filtered using a 5 micron nylon filter cloth to remove residual NaCl. This provided BMX-001-1 [13.4 kg, 85% yield (corrected for THF content), 2.5 wt % THF] as a pale yellow liquid.

In this example, the equivalents of reagents and solvents were optimized to maximize the conversion and yield of BMX-001-1.

During the workup stage, the organic solvent must be removed in order to wash away residual 2-butoxyethanol with water washes. 2-butoxyethanol will not partition into the aqueous layer in the presence of organic solvents (MTBE, THF, CH₂Cl₂, EtOAc, IPAc, and heptane).

While not wishing to be bound to any particular theory, the amount of residual THF in BMX-001-1 may be a relevant process parameter. THF will react with BMX-001-1 under the reaction conditions used in the next step and generate an impurity in BMX-001-2-Cl which is difficult to remove. In order to minimize this impurity, the amount of THF in BMX-001-1 should be less than 1 weight percent (relative to BMX-001-1).

The 2-butoxyethanol may contain an impurity of methanol and thus methyltoluenesulfonate will be formed. As methylation is extremely fast (due to the lack of steric issues with small methyl group) even a tiny amount of methanol will result in the production of a small amount of an impurity with three butoxyethyl chains and one methyl chain.

### Examples 2-3

### Synthesis of BMX-001-2-PF₆ and BMX-001-2-Cl

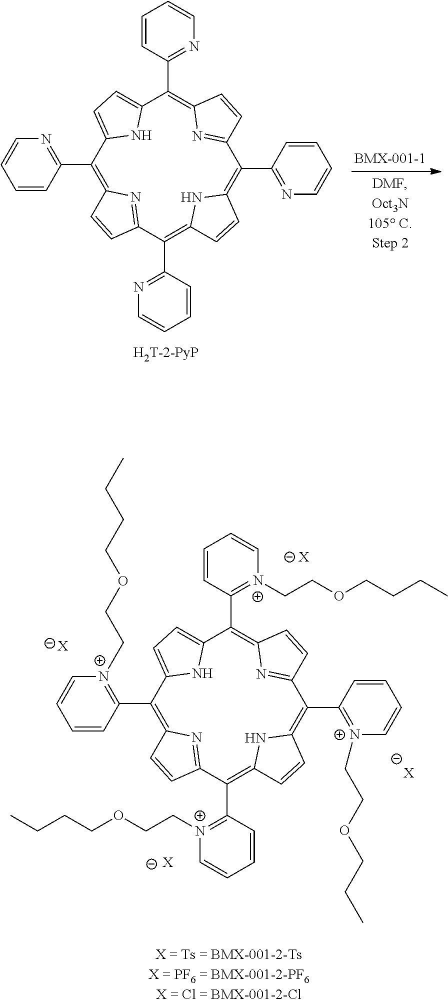

### Example 2

### Synthesis of BMX-001-2-PF₆

A solution of H₂T-2-PyP (100 g, 161.6 mmol), tri-n-octylamine (Oct₃N) (572 g, 1.62 mol) and N,N-dimethylformamide (DMF) (6.0 L) were sparged with N₂ for 15 minutes and then heated to 80° C. (internal temperature). At 80° C., the sparge tube was removed and the batch placed under a slow sweep of N₂. The batch was heated to 105° C. and BMX-001-1 (8.8 kg, 32.3 mol, containing 2.5 wt % THF) was added while maintaining 85-105° C. After the addition was complete, the batch was reheated to 105° C. The progress of the reaction was monitored by HPLC. After 45 hours, the reaction was deemed complete by HPLC. The reaction was cooled to room temperature and filtered through a thin pad of Solka Floc on top of an 18 inch (11 micron) sharkskin filter paper. The filtrate was then added slowly over 75 minutes to a flask containing a mixture of Solka Floc 40NF (1.0 kg, International Fiber) and MTBE (60 L). After the addition was complete, the slurry was stirred for 15 minutes and then filtered using 18 inch (11 micron) sharkskin filter paper.

The BMX-001-2-OTs containing Solka Floc solids were washed with a 1/1 solution of THF (2.5 L) and MTBE (2.5 L). The Solka Floc solids were then dried under vacuum at room temperature for 20 hours.

The crude BMX-001-2-OTs was rinsed off of the Solka Floc using water (10 L, House RO water). The filtrate was treated with DARCO G60 activated charcoal (40 g) and stirred for 1 hour at room temperature. The mixture was then filtered through a thin pad of Solka Floc 40NF to provide an aqueous solution of BMX-001-2-OTs.

The aqueous BMX-001-2-OTs solution was treated with saturated aqueous Brine (2.5 L). The batch was transferred to a 22 L flask and a solution of NH₄PF₆ (200 g) in water (600 mL, House RO water) was added slowly over 60 minutes. The resulting red slurry was stirred for 65 minutes and then filtered using a 10 inch nutsche with a 5 micron nylon filter cloth. The solids were dried under vacuum on the filter with N₂ applied to the top of the cake for 41 hours. This provided BMX-001-2-PF₆ [217 g, 84% yield, 88.7% (AUC) by HPLC, 2.7 wt % water] as a red solid.

In this example, the volumes of solvent (DMF), equivalents of BMX-001-1, and equivalents of Oct₃N have been optimized to maximize conversion of H₂T-2-PyP to BMX-001-2 and to minimize the formation of impurities during prolonged heating at 105° C.

In addition, the feature of isolating the BMX-001-2-OTs by precipitation onto solka floc serves to reduce “oiling out” of the intermediate onto reactor walls. Extraction of BMX-001-2-OTs from solka floc with water and direct conversion to BMX-001-2-PF₆ helps to avoid problematic aqueous workup where the product partitions into both aqueous and organic phases. Incorporation of a charcoal treatment and addition of NaCl also help reduce oiling-out during the precipitation of BMX-001-2-PF₆.

### Example 3

### Synthesis of BMX-001-2-Cl

BMX-001-2-PF₆ (200 g) was charged to a 50 L glycol jacketed glass reactor. To the reactor was added acetone (10.0 L) and the mixture was stirred until the solids dissolved. Methyl isobutyl ketone (MIBK) (10.0 L) was then added to the reactor and the batch was stirred for 15 minutes. A solution of Aliquat 336 (441 g Alfa Aesar) in acetone (2.0 L) and MIBK (2.0 L) was added drop wise to the batch over 65 minutes under a nitrogen atmosphere. This produced a fine slurry of red solids. After stirring for an additional 30 minutes, the slurry was filtered using a 10 inch nutsche with a 5 micron nylon filter cloth. The mixture was kept under positive pressure of N₂ during the filtration to avoid moisture contamination. The solids were then washed with a mixture of 1/1 acetone/MIBK (2×10.0 L) and dried for 17 hours under vacuum with N₂ applied to the top of the cake. This provided BMX-001-2-Cl [147 g, 100% yield, 88.7% (AUC) by HPLC] as a red solid.

In this example, MIBK is used as a less hazardous alternative to Et₂O as the antisolvent for precipitating BMX-001-2-Cl. In addition, Aliquat® 336 is used instead of nBu₄NCI to exchange the PF₆ anion for the Cl anion. Aliquat® 336 has better solubility in acetone and MIBK, and is easier to wash away during isolation of BMX-001-2-Cl.

### Example 4

### Optional Purification of BMX-001-2-Cl

Plug Column:

BMX-001-2-Cl (145 g) was purified by a silica gel (1.5 kg, Silicycle) plug column using 1/3/3 sat. aqueous KCl (Fisher)/Water (House RO water)/CH₃CN (Fisher7) as the eluent. Seven dark red colored fractions were collected and analyzed by HPLC. The first three fractions (≥88.9% AUC) were combined and concentrated to ⅓ of the original volume to remove CH₃CN. The mixture was diluted with water (12.0 L, House RO water) and transferred to a 22 L reactor. A solution of NH₄PF₆ (300 g, SynQuest) in water (900 mL, House RO water) was added slowly to the batch over 60 minutes. The resulting purple slurry was stirred for 30 minutes and then filtered using a 10 inch nutsche with a 5 micron nylon filter cloth. The solids were dried on the filter under vacuum for 62 hours. This provided BMX-001-2-PF₆ [155 g, 75% yield, 90.4% (AUC) by HPLC] as a red-purple solid.

Conversion of BMX-001-2-PF₆ Back to BMX-001-2-Cl:

BMX-001-2-PF₆ (153 g) was added to a 22 L reactor. Acetone (7.65 L) was added and the mixture stirred until the solids dissolved. MIBK (7.65 L, Pharmco) was then added to the reactor and the batch was stirred for 15 minutes. A solution of Aliquat® 336 (337 g, Alfa Aesar) in acetone (1.5 L, SAFC) and MIBK (1.5 L, Pharmco) was added drop wise to the batch over 70 minutes under a nitrogen atmosphere. This produced a fine slurry of red solids. After stirring for an additional 30 minutes, the slurry was filtered using a 10 inch nutsche with a 5 micron nylon filter cloth. The mixture was kept under positive pressure of N₂ during the filtration to avoid moisture contamination. The solids were then washed with a mixture of 1/1 acetone (SAFC)/MIBK (Pharmco) (2×7.7 L) and dried for 16 hours under vacuum with N₂ applied to the top of the cake. This provided BMX-001-2-Cl [120 g, 100% yield, 90.4% (AUC) by HPLC] as a red solid.

Optional SiO₂ chromatography with KCl, CH₃N, and water can be used to increase purity of BMX-001-2-Cl by about 1 to 2 percent (AUC by HPLC). KCl is important for this chromatography. KCl causes the porphyrin to form aggregates and travel through the stationary phase as a single band. Without KCl, the material does not elute from the stationary phase.

### Examples 5-8

### Synthesis of BMX-001-PF₆ and BMX-001

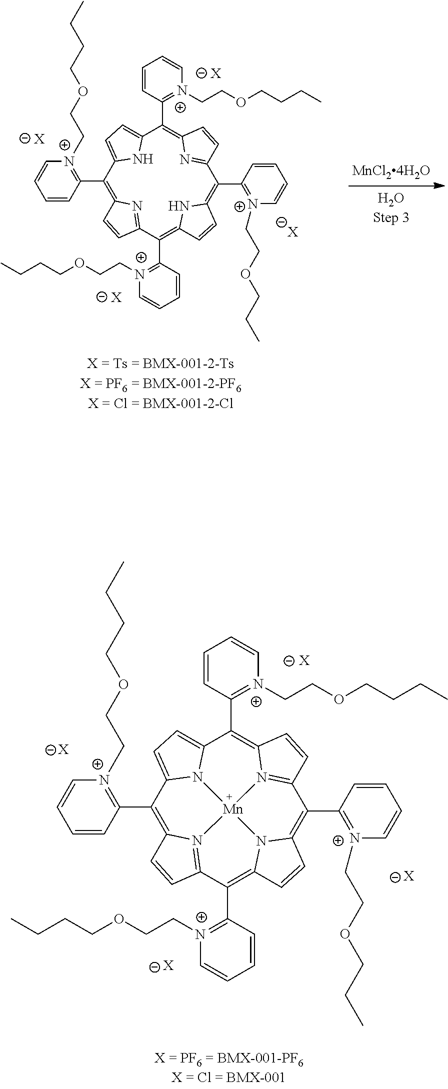

### Example 5

### Synthesis of “Crude” BMX-001-PF₁

BMX-001-2-Cl (120 g) was added to a 22 L reactor followed by water (12.0 L, House RO water). The mixture was stirred for 15 minutes and then the pH was adjusted to pH=11.0 using aqueous 1 M NaOH. To this solution was added MnCl₂×4 H₂O (306 g, SAFC) in a single portion and the resulting mixture was stirred at ambient temperature. After adding MnCl₂×4 H₂O, air was bubbled through the batch using a inch polypropylene tube at a flow rate of 0.1 cfm. The pH of the batch was monitored and adjusted to pH 8.0 by adding additional 1 M NaOH every 30 minutes for the first two hours. The reaction was monitored by HPLC to determine both metal insertion and oxidation of the intermediate Mn(II) porphyrin to the desired Mn(III) porphyrin. After 5 hours, the reaction was deemed complete. The mixture was filtered using a 10 inch nutsche (5 micron nylon filter cloth) and a pad of solka floc 40NF.

The filtrate was transferred to a 22 L reactor and a solution of NH₄PF₆ (270 g, SynQuest) in water (810 mL, House RO water) was added slowly to the batch over 60 minutes. The resulting red slurry was stirred for 35 minutes and then filtered using a 10 inch nutsche with a 5 micron nylon filter cloth. The solids were dried on the filter under vacuum for 40 hours. This provided BMX-001-PF₆ [174 g, 94% yield, 89.4% (AUC) by HPLC, 2.9 wt % water] as a red solid.

While not wishing to be bound to any particular theory, it is believed that initial pH adjustment to pH 11 may help achieve rapid and clean metalation of BMX-001-2-Cl with MnCl₂×4 H₂O. The equivalents of MnCl₂×4 H₂O are optimized (15 equiv) to minimize residual Mn in BMX-001. While not wishing to be bound to any particular theory, pH control during metalation may help in ensuring smooth metalation and/or subsequent air oxidation of the Mn(II) intermediate to the desired Mn(III) oxidation state of BMX-001.

### Example 6

### Synthesis of “Crude” BMX-001

BMX-001-PF_{G} (170 g) was added to a 22 L reactor. Acetone (7.65 L, SAFC) was added and the mixture stirred until the solids dissolved. MIBK (7.65 L, Pharmco) was then added to the reactor and the batch was stirred for 15 minutes. A solution of Aliquat® 336 (334 g, Alfa Aesar) in acetone (1.5 L, SAFC) and MIBK (1.5 L, Pharmco) was added drop wise to the batch over 100 minutes under a nitrogen atmosphere. This produced a fine slurry of red solids. After stirring for an additional 75 minutes, the slurry was filtered using a 10 inch nutsche with a 5 micron nylon filter cloth. The mixture was kept under positive pressure of N₂ during the filtration to avoid moisture contamination. The solids were then washed with a mixture of 1/1 acetone (SAFC)/MIBK (Pharmco) (2×8.5 L) and dried for 22 hours under vacuum with N₂ applied to the top of the cake. This provided BMX-001 [117 g, 99% yield, 89.4% (AUC) by HPLC] as a brown solid.

In this example, BMX-001 was converted to BMX-001-PF₆ and then back to BMX-001 to reduce the level of residual manganese. This example again included the use of MIBK instead of Et₂O (hazardous) as the antisolvent for precipitating BMX-001, and used Aliquat® 336® instead of nBu₄NCl to exchange PF₆ anion for Cl anion.

### Example 7

### Synthesis of BMX-001-PF₆

BMX-001-2-C1 (110 g) was added to a 22 L reactor followed by water (8.8 L, House RO water). A solution of NH₄PF₆ (248 g, SynQuest) in water (743 mL, House RO water) was added slowly to the batch over 60 minutes. The resulting red slurry was stirred for another 40 minutes and then filtered using a 10 inch nutsche with a 5 micron nylon filter cloth. The solids were washed with water (2×1.0 L, House RO water) and then dried on the filter under vacuum for 68 hours. This provided BMX-001-PF₆ [145 g, 92% yield, 89.1% (AUC) by HPLC, 3.7 wt % water] as a red solid.

While not wishing to be bound to any particular theory, this additional precipitation may help to reduce the amount of free residual Mn mixed with BMX-001.

### Example 8

### Synthesis of BMX-001

BMX-001-PF₆ (140 g) was added to a 22 L reactor. Acetone (6.3 L, SAFC) was added and the mixture stirred until the solids dissolved. MIBK (6.3 L, Pharmco) was then added to the reactor and the batch was stirred for 15 minutes. A solution of Aliquat® 336 (275 g, Alfa Aesar) in acetone (1.3 L) and MIBK (1.3 L, Pharmco) was added drop wise to the batch over 90 minutes under a nitrogen atmosphere. This produced a fine slurry of red solids. After stirring for an additional 45 minutes, the slurry was filtered using a 10 inch nutsche with a 5 micron nylon filter cloth. The mixture was kept under positive pressure of N₂ during the filtration to avoid moisture contamination. The solids were then washed with a mixture of 1/1 acetone (SAFC)/MIBK (Pharmco) (2×7.0 L) and dried for 44 hours under vacuum with N₂ applied to the top of the cake. The BMX-001 solids were transferred to a glass tray and dried inside of a vacuum oven until constant mass was reached. Note: The solids were dried at ambient temperature and a slow bleed of N₂ was introduced into the oven to help clear the headspace of solvent vapors. After 95 hours inside the vacuum oven, drying until constant mass was reached. This provided BMX-001 [96.7 g, 100% yield, 89.99% (AUC) by HPLC] as a brown solid.

While not wishing to be bound to any particular theory, this additional precipitation step may help to reduce the amount of free residual Mn mixed with the BMX-001.

### Example 9

Described below are studies that preceded and guided the preparation of the metal-based, redox-active therapeutic Mn(III) meso-tetrakis(N-n-butoxyethylpyridyl)porphyrin, MnTnBuOE-2-PyP⁵⁺ (BMX-001), which is currently in Phase I/II Clinical Trials as a radioprotector of normal tissue in cancer patients. N-substituted pyridylporphyrins are ligands for Mn(III) complexes that are among the most potent superoxide dismutase (SOD) mimics thus far synthesized. To advance their design, thereby improving their physical and chemical properties and bioavailability/toxicity profiles, a systematic study on placing oxygen atoms into N-alkylpyridyl chains via alkoxyalkylation reaction was undertaken. Shown herein are the unforeseen structural rearrangement that happens during the alkoxyalkylation reaction by the corresponding tosylates. Comprehensive experimental and computational approaches were employed to solve the rearrangement mechanism involved in quaternization of pyridyl nitrogens, which, instead of a single product, led to a variety of mixed N-alkoxyalkylated and N-alkylated pyridylporphyrins. The rearrangement mechanism involves the formation of an intermediate alkyl oxonium cation in a chain-length-dependent manner, which subsequently drives differential kinetics and thermodynamics of competing N-alkoxyalkylation versus in situ N-alkylation. The use of numerous alkoxyalkyl tosylates, of different length of alkyl fragments adjacent to oxygen atom, allowed us to identify the set of alkyl fragments that would result in the synthesis of a single compound of high purity and excellent therapeutic potential.

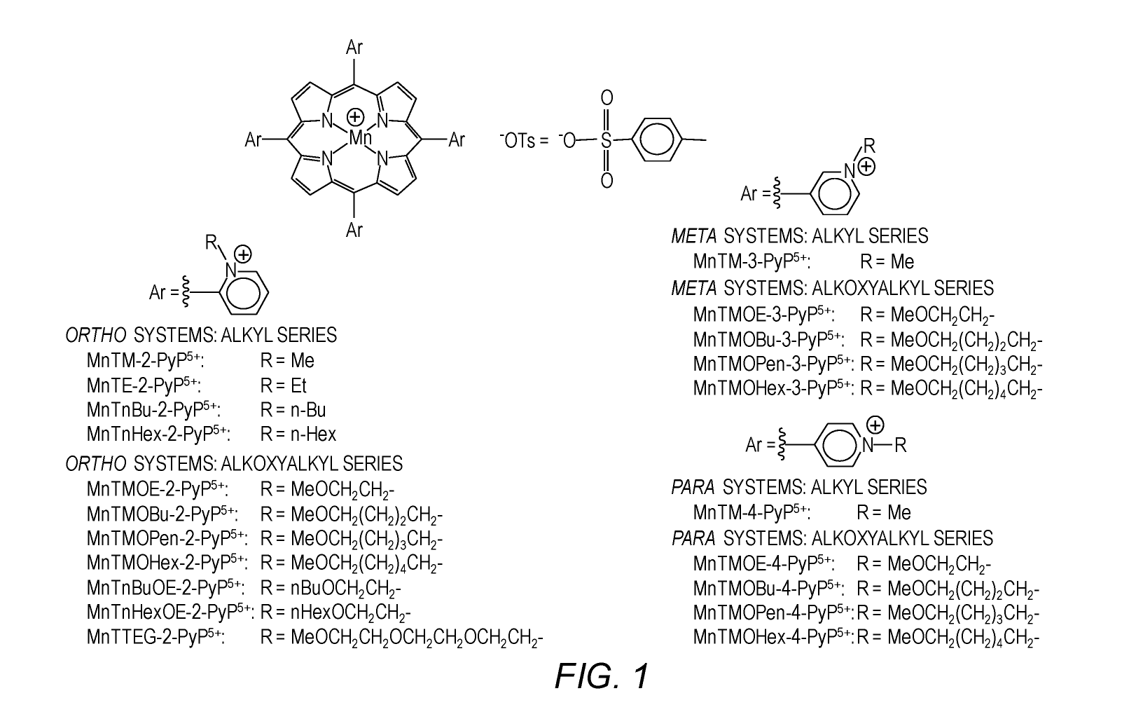

Cationic Mn(III) porphyrins are among the most efficacious SOD mimics and redox-active experimental therapeutics for the treatment of diseases associated with a disturbed cellular redox environment, commonly described as a state of oxidative stress. Among N-alkyl-substituted pyridyl- or imidazolyl Mn porphyrin, their ortho isomers are the most studied compounds in vitro and in vivo. These include Mn(III) meso-tetrakis-(N-ethylpyridinium-2-yl)porphyrins (MnTE-2-PyP⁵⁺, AEOL10113, BMX-010), Mn(III) meso-tetrakis-(N,N′-diethylimidazolium-2-yl)porphyrin (MnTDE-2-ImP⁵⁺, AEOL10150), Mn(III) meso-tetrakis-(N-n-hexylpyridinium-2-yl)porphyrin (MnTnHex-2-PyP⁵⁺), and, more recently, Mn(III) meso-tetrakis-(N-n-butoxyethylpyridinium-2-yl)porphyrin (MnTnBuOE-2-PyP⁵⁺, BMX-001) (FIG. 1).

The development of redox-active therapeutics has paralleled the advances in synthesis of powerful SOD mimics. Mn(III) 2-N-alkylpyridylporphyrins emerged as potent SOD mimics, some of which approaching the activity of SOD enzymes. Whereas the intrinsic antioxidant potency of MnPs is physico-chemically controlled, their biological activity relies also on their toxicity, and bioavailability, which, in turn, depends on factors such as size and lipophilicity. The understanding of key structural features of MnPs in controlling intrinsic SOD activity, compound stability, lipophilicity, bioavailability, sub-cellular localization, and pharmacokinetics have paved the way to the optimization of other related compounds.

The optimization of MnP-based therapeutics has been actively sought by the controlled modification of the side-chain pyridinium moieties. Short alkyl-chained analogues, such as MnTE-2-PyP⁵⁺, are of low lipophilicity and therefore low availability to brain tissue, which limits its use in the treatment of central nervous system disorders. Nonetheless, successful pre-clinical profile of the short alkyl-chained derivative MnTE-2-PyP⁵⁺ in a series of disease models has forwarded it into Phase I/II Clinical Trials in Canada. Long alkyl-chained analogues, such as MnTnHex-2-PyP⁵⁺, accumulate in cells at higher levels than its ethyl analogue. Yet, systemic administration of the lipophilic N-alkylpyridylporphyrins is hampered by toxicity associated with their surfactant/micellar properties. As an attempt to reduce the surfactant character brought by the long alkyl side chains, a strategy of replacing a CH₂ group of the alkyl chains by oxygen atoms to yield alkoxyalkyl analogues was envisaged. Yet the actual execution of such approach was troublesome.

Described herein are the pitfalls that hampered those studies and the experimental and computational studies that eventually guided us into the development of remarkable SOD mimic—MnTnBuOE-2-PyP⁵⁺ (FIG. 1). The notable biological efficiency and safe toxicity profile (e.g., lack of genotoxicity in a rat Comet assay) of MnTnBuOE-2-PyP⁵⁺ in pre-clinical studies have justified its pursuit toward clinics; indeed, MnTnBuOE-2-PyP⁵⁺ is now in Phase I/II Clinical Trials on glioma patients (NCT02655601) as a radioprotector of normal brain and will enter soon another trial on radioprotection of salivary glands and mouth mucosa with head & neck cancer patients. More specifically, it is shown herein that the impurities hampering the development of oxygenated side-chain MnPs, such as methoxyalkyl (MOalkyl) derivatives (alkyl=Et, n-Bu, n-Pen, and n-Hex) of ortho, meta, and para Mn(III)N-pyridylporphyrins relate to the unexpected formation of methyl-containing MnPs. The extent of contamination varied with the length of the methoxyalkyl chains and limited severely the use of some of the methoxyalkyl constructs, as separation of methyl- and methoxyalkyl-containing species is difficult. This, in turn, compromises biological testing of the samples. Understanding of the nature and origin of these impurities, which plagued all methoxyalkyl MnP preparations, will facilitate future synthetic endeavors in the field of lipophilic, non-toxic MnP-based therapeutics. The mechanism associated with competing methylation/methoxyalkylation reactions was studied by Density Functional Theory at the M06-2X level and correlated well with the experimental data. The overall results presented here calls for a reevaluation of the previously published PEG and methoxyalkyl data on both Fe(III) and Mn(III) porphyrins, such as FP-15, MnTTEG-2-PyP⁵⁺, and MnTMOE-2-PyP⁵⁺.^{25,26}

### Materials and Methods

Reagents.

H₂T-2-PyP, H₂T-3-PyP and H₂T-4-PyP were purchased from Frontier Scientifi, 2-Methoxyethyl tosylate (>98%), 4-methoxybuthanol (>98%), 6-bromohexan-1-ol (>95%), from TCI America, 5-methoxypenthanol (98%) from Karl Industries Inc., p-toluenesulfonyl chloride (98%) from Alpha Aesar, pyridine (99%) and tetra-n-butylammonium chloride hydrate (98%) from Aldrich, MnCl₂×4H₂O (99.7%) and hexane from J. T. Baker and NH₄PF₆ (99.99% pure) from GFS chemicals. Diethyl ether anhydrous and acetone were from EMD chemicals, absolute methanol, ethyl acetate, dichloromethane, chloroform, acetonitrile, EDTA and KNO₃ from Mallinckrodt, 98% anhydrous N,N-dimethylformamide (kept over 4-Å molecular sieves), plastic-backed silica gel TLC plates (Z122777-25EA) from Sigma-Aldrich and silica (SiliaFlash® G60, 70-230 mesh) from Silicycle (Canada). 6-Methoxyhexan-1-ol and H₂TMOE-2-PyP⁴⁺ were prepared as previously described.^{26,27} All other chemicals were used as received. 4-Methoxybutyl, 5-methoxypentyl, and 6-methoxyhexyl tosylates. Syntheses were carried out as described earlier.^{28,29} In short, to a 50 mL CHCl₃ solution containing 0.048 mol of the appropriate methoxyalcohol (4-methoxybutanol: 5.00 g; 5-methoxypentanol: 5.67 g; 6-methoxyhexanol: 6.40 g) at 0° C., pyridine (7.763 mL, 0.096 mol) was added, followed by the dropwise addition of a 50 mL CHCl₃ solution of p-toluenesulfonyl chloride (13.73 g, 0.072 mol).The reaction mixture was stirred at 0° C. for 2 h (for 4-metoxybutanol and 5-methoxypentanol) or 4.5 h (for 6-methoxyhexanol). After extraction with H₂O (4×100 mL), 2 M HCl (4×100 mL), saturated NaHCO₃ solution (till pH ˜6) and H₂O (3×100 mL), the organic phase was dried with anhydrous Na₂SO₄ and filtered. The solution was evaporated in a rotary evaporator and the oily residue was purified by flash chromatography (CombiFlash instrument, mobile phase=Hex:EtOAc). The fractions contained the desired product were combined and evaporated on a rotary evaporator to yield a colorless oil. ¹H, ¹³C NMR, and MS data were in agreement with the proposed structures. Yield: 4-methoxybutyl tosylate: 84.7% (10.50 g); 5-methoxypentyl tosylate: 76.7% (10.03 g); 6-methoxyhexyl tosylate: 90% (12.37 g).

Mn porphyrins.

The methoxyalkylation of H₂T-X-PyP (X=2, 3, or 4) and the subsequent Mn metallation to prepare MnTMOE-X-PyPCl₅, MnTMOBu-X-PyPCl₅, MnTMOPen-X-PyPCl₅, and MnTMOHex-X-PyPCl₅ (X=2, 3, or 4) were carried out as previously described for other related alkyl systems.³⁰ To a solution of H₂T-X-PyP (X=2, 3, or 4) (20 mg, 0.032 mmol) in anhydrous DMF (2 mL, preheated at 105° C. for 15 min) the appropriate tosylate was added (2-methoxyethyl tosylate, MeOEtOTs: 3.67 g, 0.016 mol; 4-methoxybutyl tosylate, MeOBuOTs: 4.18 g, 0.016 mol; 5-methoxypentyl tosylate, MeOPenOTs: 4.00 g, 0.016 mol; 6-methoxyhexyl p-tosylate, MeOHexOTs: 2.20 g, 0.008 mol). The course of the reaction was followed by TLC, using 1:1:8 KNO₃-saturated H₂O:H₂O:acetonitrile as mobile phase. The reaction mixture was filtrated into a separatory funnel containing H₂O and chloroform and extracted several times with chloroform. The isolation of chloride salt, the metalation with MnCl₂ and the isolation of Mn porphyrin as chloride salt was carried out as previously described for Mn(III)N-alkylpyridylporphyrins.³⁰ The products were dried under vacuum at room temperature. Isolated solids were labeled “methoxyalkyl chain/porphyrin isomer” according to the starting methoxyalkyl tosylate and porphyrin used; a short form was used for both the tosylates (i.e., MeOEt, MeOBu, MeOPen, MeOHex) and porphyrin isomer (2-Py, 3-Py, and 4-Py standing for ortho, meta, and para N-pyridylporphyin systems), respectively. Drying the solids at high temperature was not attempted in order to avoid likely thermal dealkylation, as reported previously for related MnTE-2-PyP.³¹ It is worth noting that TLC and ESI-MS analyses indicate that solids are fully quaternized but are not single compounds (see Results and Discussion Section).

Analysis of the Mn Complexes.

Electrochemistry, electrospray-ionization mass spectrometry (ESI-MS), UV-visible spectroscopy and SOD-like activity were carried out as previously described.^{29, 32}. All quantum chemistry calculations have been performed at the M06-2X/6-311++G(2d,p)//M06-2X/6-31+G(d) DFT level³³⁻³⁸ using the Gaussian 09 software.³⁹ All frequency calculations were carried out at 105° C. and used to characterize minima and transition states. The solvent effect has been taken into account using the CPCM continuum solvation model⁴⁰ for N,N-dimethylformamide (DMF). The free energies of reactants, transition states, and products have been obtained from the ideal gas partition functions for the structures optimized in solution⁴⁰ and corrected to include the compression work of the gas⁴¹ (or liberation free energy⁴²) to standard 1 mol L⁻¹ concentration. The coulombic stabilization energy due to the formation of ionic pairs in DMF has also been included in the final results by approximating each ion as a sphere, whose volume was considered the same as that of the solute cavity;⁴³ the distance between cation and anion in the ionic pair was taken as the sum of the two sphere radii.

Results and Discussion

Quartenization of Mn(III)N-pyridylporphyrins with methoxyalkyl tosylates is compromised by competing in situ methylation. The introduction of oxygenated alkyl side-chains has been explored as a means to reduce the toxicity of Mn(III)N-alkylpyridylporphyrins. We describe here the synthetic drawbacks associated with this synthetic strategy. Gaining insights into the synthetic approaches benefited the development of MnTnBuOE-2-PyP⁺ paving its pathway towards clinic. The methoxyalkylation of all three isomers of N-pyridylporphyrins was carried out with four tosylates of appropriate chain length (i.e., MeOEtOTs, MeOBuOTs, MeOPenOTs, and MeOHexOTs), accounting for 12 preparations. The synthetic and purification routes were adapted from that of related alkyl derivatives and involved the reaction of H₂T-X-PyP (X=2, 3, or 4) with the appropriate tosylate in DMF at 105° C. followed by metallation with MnCl₂ under aqueous conditions at room temperature. None of the isolated solids appeared to be a single compound (see below).

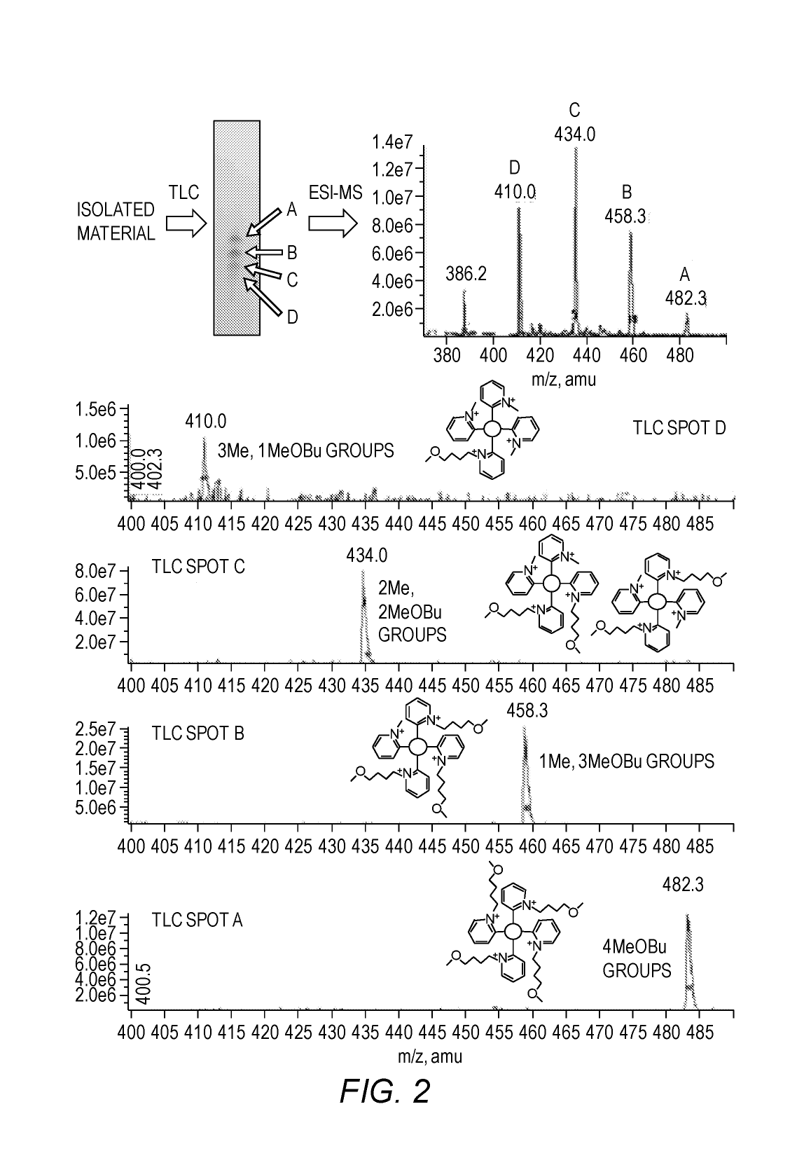

Methoxyalkylation reactions were monitored by TLC. None of the preparations yielded a single TLC spot. Indeed, TLC plates showed that some preparations were a mixture of at least 5 products almost evenly distributed. Reactions were deemed complete when the starting porphyrin had been fully consumed and the resulting spotting profile did not change with time. Whereas the presence of more than one TLC spot is common for ortho isomers bearing long alkyl chains (e.g., n-hexyl), as a result of them being a mixture of atropisomers, a single TLC spot has always been observed in the case of ortho isomers with short alkyl chains (e.g., methyl and ethyl), as well as for meta and para isomers, for which atropisomerism is not expected. The origin of TLC spots as a result of incomplete quartenization of MnPs, was ruled out based on the following evidences: (a) prolonged heating time and additional amounts of the methoxyalkylating reagents did not change the TLC profile; (b) the UV-vis spectra of the isolated materials were characterized by a well-defined Soret band in a region expected for fully quaternized MnPs (partial quaternization would have shifted the Soret band to higher wavelengths); (c) voltammograms of the isolated MnPs were symmetrical and with no shoulders, indicating either the presence of only one MnP species (which is not the case, given the TLC analyses), or that the sample contains a mixture of very closely related species with nearly identical Mn(III)/Mn(II) metal-centered reduction potential, E. A mixture of species of varying degree of quaternization would have yielded ill-defined voltammograms, which was not observed in any of the preparations. The ESI-MS spectra showed a set of peaks, typical of a mixture of compounds and consistent with TLC data. Heptafluorobutyrate anion (HFBA⁻) was used as ion-pairing agent for ESI-MS analysis under conditions which excluded MnP fragmentation, as reported elsewhere. The ESI-MS spectra of all samples were characterized by two sets of peaks (FIG. 2). The first set ranging from m/z 385 to m/z 520 occurs in the region regularly associated with the ion-paired cluster (MnP⁵⁺+2HFBA⁻)³⁺/3, whereas the second set at m/z 685-890 relates to the ion-pair (MnP⁵⁺+3HFBA⁻)²⁺/2. Although the expected peaks corresponding to the fully quaternized methoxyalkylated species were observed in each case, these peaks were always accompanied by other peaks of lower m/z values and sometimes of greater intensity. A breakthrough in characterizing these systems was achieved by coupling the ESI-MS analysis with pre-separation of the samples by TLC-SiO₂ (sat. KNO_{3(aq)}:H₂O:CH₃CN, 1:1:8 v/v/v). Each TLC spot was isolated, and the MnP recovered from each TLC spot was individually analyzed by ESI-MS. As a typical case, TLC analysis of the “MeOBu/3-Py” material gave rise to 4 spots. Upon ESI-MS analysis 4 clean spectra characteristic of four MnP⁵⁺-type compounds were obtained, whose spectral features, corresponding to the (MnP⁵⁺+2HFBA⁻)³⁺/3 ion-pairing region, are shown in FIG. 2. It is worth noting that each spectrum in FIG. 2 contains 1 of the four peaks in the m/z 385-520 region of the originally isolated sample; the same is true for other spectral regions (not shown). The ESI-MS data for each compound in FIG. 2 are consistent with a fully quarternized MnP⁵⁺ species (ion-paired with 2 HFBA⁻ anions) in which both the number of methoxyalkyl moieties decreased from 4 to 1 and the number of methyl groups increased correspondingly from 0 to 3, maintaining the total number of substituents at the pyridyl moieties equals to four. The greater the number of methyl groups in the sample, the smaller the TLC R_{f} value, which was consistent with previous data on the increase in MnP⁵⁺ polarity (reduced lipophilicity) with decrease in length of pyridyl side-chain.^{11, 12, 30} In the related ortho system, “MeOBu/3-Py” (FIG. 2), the extra peaks at m/z 386.2 and at m/z 685.5, correspond unambiguously to a well characterized compound containing no methoxybutyl groups at all, but 4 methyl groups instead, i.e., MnTM-3-PyP⁵⁺. Similar scenario was seen with MeOBu/2-Py system. It is worth noting that elemental analysis and C/N ratios of some of the isolated solids were surprisingly fine. Thus, based on elemental analysis, there was not a hint on how far away from a single compound some of the preparations were.

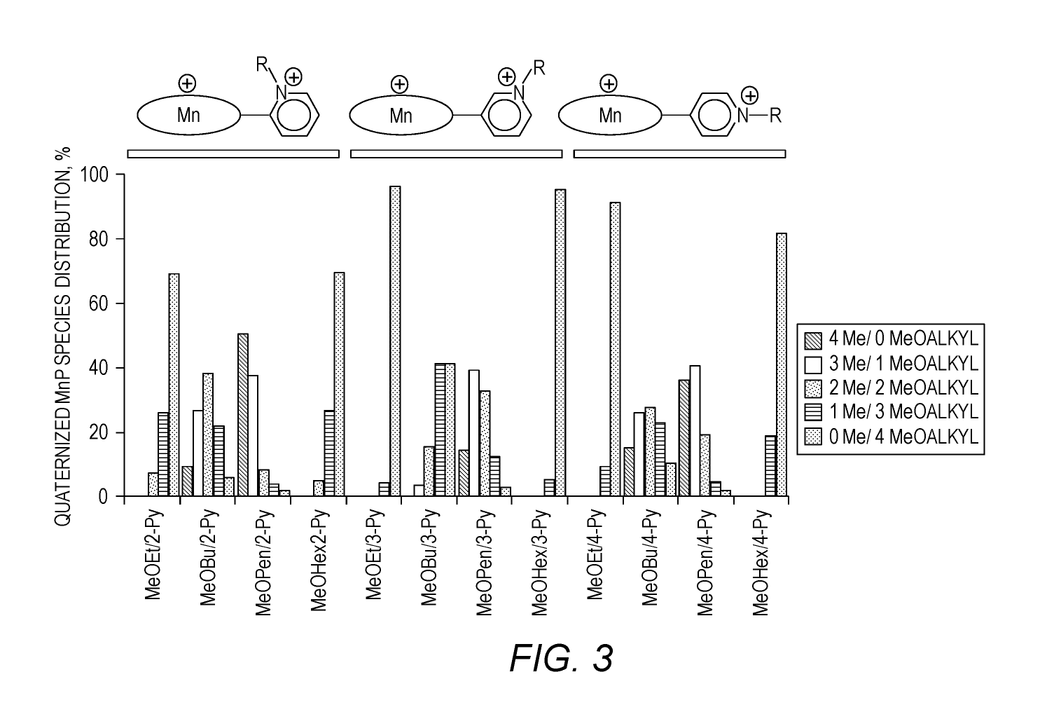

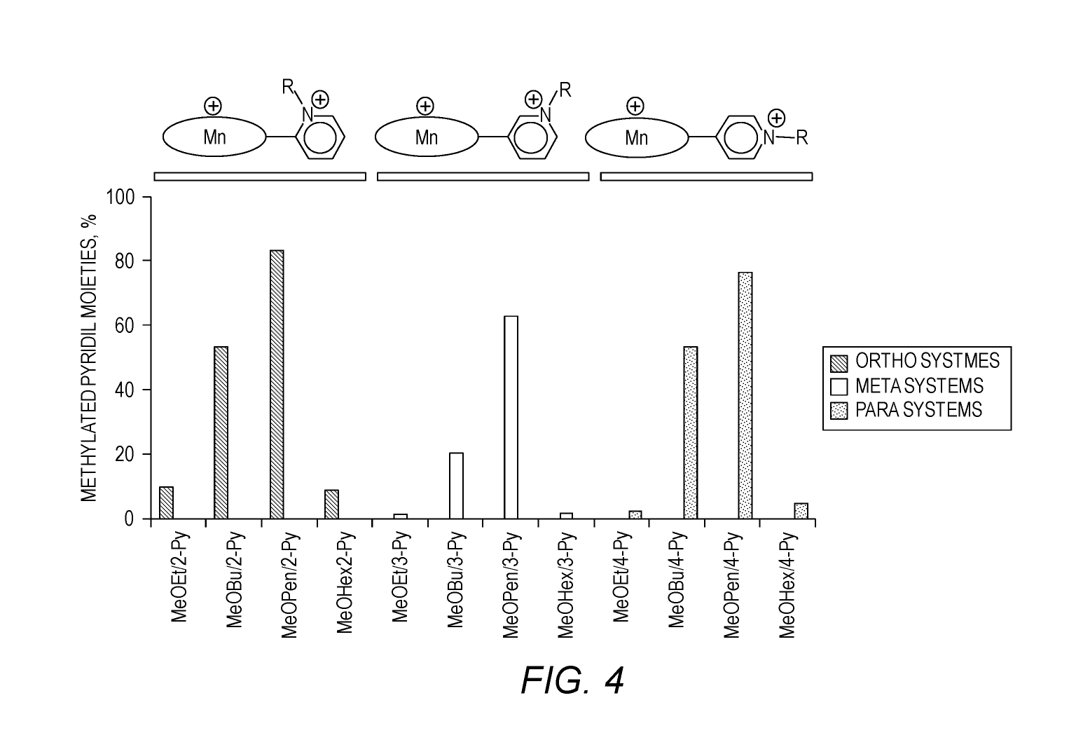

Considering that all MnP⁵⁺ species, regardless of the type of side-chain being methyl and/or methoxyalkyl, should share similar features related to ionization, ion-pairing, and ion suppression behavior, the intensities of the peaks in the ESI-MS spectra were used as a crude measure of the contribution of each individual species to a whole isolated mixture. The distribution ratio of the desired tetramethoxyalkylated porphyrin against the side-products in which pyridyl groups had instead been quaternized by one, two, three, or four methyl groups in each of the 12 preparation is depicted in FIG. 3. The degree of overall methylation that took place in detriment of methoxyalkylation is presented in FIG. 4. The ESI-MS data in FIG. 2 agree with the relative color intensity of the TLC spots, as judged qualitatively by visual inspection. The examination of the distribution data (FIG. 3) and the degree of methylation versus methoxyalkylation (FIG. 4) indicated that the feasibility and extent of methylation varied with the nature of both the porphyrin isomer and the methoxyalkyl tosylate used. The general trends in these systems are summarized as it follows: (i) unwanted methylation is more pronounced in the ortho isomer systems than in the meta or the para ones; (ii) methoxyalkylation is favored (as opposed to methylation) by the use of MeOEtOTs and MeOHexOTs, whereas methylation prevails and the target tetramethoxyalkylated MnP is minimal with the use of MeOBuOTs and MeOPenOTs; (iii) methylation dominated the MeOPenOTs systems, with “MeOPen/X-Py” (X=2, 3, or 4) solid being particularly rich in MnTM-X-PyP⁵⁺ species (X=2, 3, or 4): the attempted methoxypentylation in the ortho system resulted in undesired MnTM-2-PyP⁵⁺ as the major compound in the isolated mixture. It is evident that methylation competes with methoxyalkylation. The source of the methyl groups in the reaction mixture and mechanistic insights into this competition are addressed below.

Mechanistic investigations: competing methoxyalkylation versus methylation. The role of the methoxyalkyl tosylate as an in situ source of both the methoxyalkyl and the methyl groups was confirmed by experimental and computational data, which helped also to shed some light on the possible mechanism(s) responsible for the competing methoxylation and methylation reactions.

Upon prolonged heating in neat DMF at 100-105° C., N-pyridylporphyrins remained unchanged, which demonstrated that the source of methyl group was neither some impurity in the batches of the starting porphyrins, nor some compound generated in situ via thermal decomposition of DMF alone. This is consistent with the overwhelming data on the preparation of the corresponding N-alkylpyridylporphyrin series (alkyl=Et, nBu, nHex, nHep, etc), in which methylation has never been observed. It could be speculated that the methylation could arise from some process involving DMF decomposition under reaction conditions in the presence of the methoxyalkyl tosylates. To unambiguously rule out the involvement of DMF as a source of methyl groups, the quaternization reactions of H₂T-2-PyP with MeOBuOTs were carried out in deuterated DMF (d₆-DMF) and the ESI-MS product distribution profile of the isolated material was identical to that observed with non-deuterated DMF. Additionally, methylation did take place but peak isotopic shifts (expected if methylation had d₆-DMF as -CD₃ group source) were not observed, which reassured the methoxyalkyl tosylates as in situ source of the methylation species.

Methoxyalkyl tosylates were thoroughly analyzed by ¹H and ¹³C NMR spectroscopy, TLC, ESI-MS and GC/MS and no impurities that could be responsible for methylation were detected. This supported a hypothesis in which the methylation species could be generated in situ via some thermal process in DMF. This represents a porphyrin-independent path. Therefore the thermal stability of the methoxyalkyl tosylates was investigated under conditions similar to the ones used in porphyrin quaternization. Thus, methoxyalkyl tosylates were heated in DMF at 100° C., while the transformations were monitored by TLC and ESI-MS. After 7 h heating, no changes were observed in the case of MeOEtOTs and MeOHexOTs. Conversely, a new product was clearly formed in the MeOBuOTs and MeOPenOTs cases. ESI-MS spectra of crude materials indicated the presence of a peak at m/z 187, which is consistent with the presence of methyl tosylate (MeOTs) in reaction mixture. TLC co-elution of these materials with an authentic MeOTs sample confirmed the formation of MeOTs upon heating of MeOBuOTs and MeOPenOTs in DMF at 100° C. Hence, the in situ formation of MeOTs could explain the competing methylation reactions observed during methoxyalkylation of the N-pyridylporphyrins.

The methoxyalkyl tosylates MeOBuOTs and MeOPenOTS were, as expected, more stable toward transformation into MeOTs at lower temperatures. At temperatures in the 60-80° C. range, MeOTs was detected upon heating MeOBuOTs and MeOPenOTS in DMF for 45 h and 21 h, respectively. Although this information is of little importance for porphyrin methoxyalkylation itself (as methoxyalkylation, alike regular alkylation, is considerably slower at these temperatures, which would allow accumulation of MeOTs and thus methylation), it establishes that MeOBuOTs is more prone to transformation into MeOTs than MeOPenOTS. This relative propensity to yield MeOTs in situ correlates with the fact that methylation prevails in the MeOPenOTs systems compared to the MeOBuOTs systems (FIG. 3). Of note, MeOTs reacts significantly faster than its longer alkyl analogues, such as Et, nBu, nHex, etc.

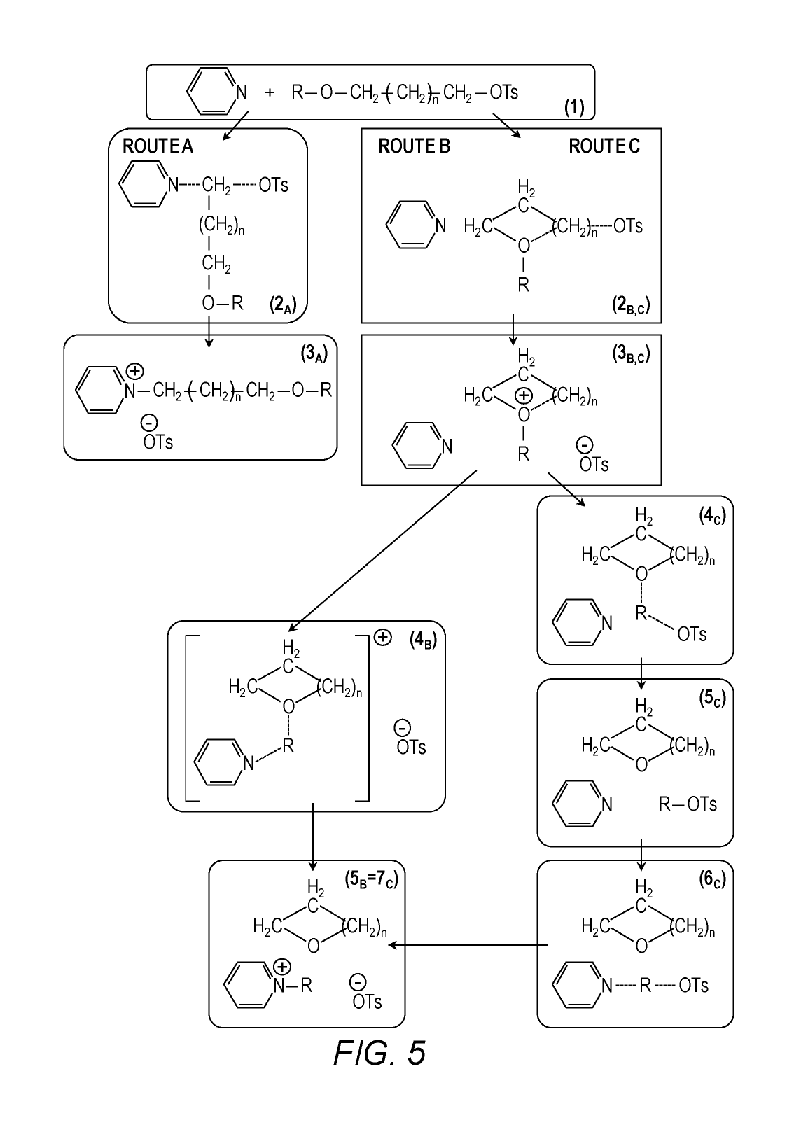

It is clear that the MnP species distribution depicted in FIG. 3 arises from the balance between two competing reactions: methoxyalkylation and methylation. Additionally, the degree of methylation given in FIG. 4 is a result of a combination of various effects, such as, the availability of unquaternized pyridyl groups, the accumulation of the in situ-generated methylating agent, and the relative reactivity of the pyridyl group toward both the methoxyalkyl tosylate and the methylating agent. Three possible routes were conceived to accommodate the net transformations observed in these systems (FIG. 5). The mechanisms and reaction profiles on each route were studied computationally in order to shed some light on the dependence of the overall competing reactivity trends on both the N-pyridylporphyrin isomer and the length of the methoxyalkyl tosylate chain. The pyridyl moieties of the N-pyridylporphyrins were represented by a free pyridine ring in FIG. 5. The use of pyridine as a surrogate for pyridyl groups is justified by that fact that each of the four pyridyl groups in the N-pyridylporphyrins reacts independently of one another; such simplification allows for more accurate calculations.

Route A (FIG. 5) depicts the mechanism associated with the desired methoxyalkylation reaction, which is suggested to follow a regular S_{N}2 mechanism via a transition state (TS) 2_{A} to yield the corresponding tosylate salt of methoxyalkylpyridinium (product 3a). The methylation reaction certainly involves a rearrangement of the methoxyalkyl tosylate to yield the methylating agent in situ, for which two complimentary routes (B and C) were envisioned: the starting methoxyalkyl tosylate rearranges into a tosylate salt of a methyl oxonium cycloalkane as a common intermediate (3_{B,C}) in both Routes B and C. This intermediate may, then, reacts directly with either pyridine (Route B) or tosylate (Route C). Route B explores the methylating properties of this oxonium salt, as there is literature precedent for alkylations carried out by trialkyloxonium salts. In Route C, the tosylate salt of methyl oxonium cycloalkane rearranges further to yield the stable products MeOTs and the corresponding cyclic ether (products 5c). The methylating agent in Route C is MeOTs, which reacts then with pyridine to yield the tosylate salt of methylpyridinium.

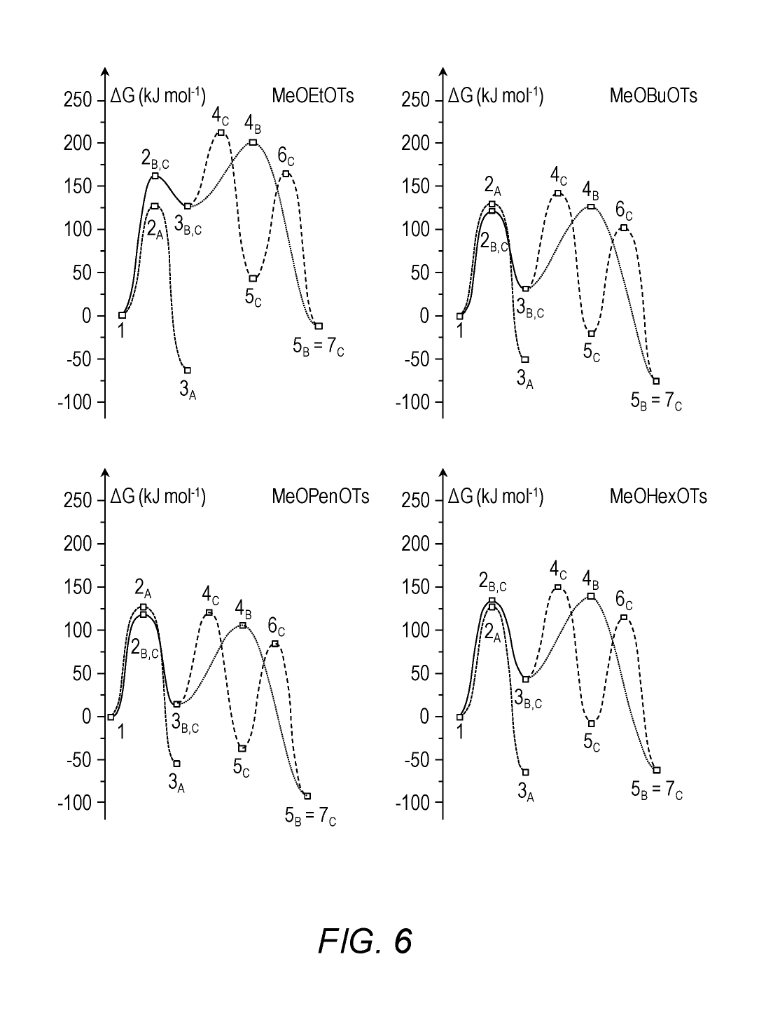

The quantum chemistry calculations on the mechanisms depicted in FIG. 5 have been performed at the DFT level using the M06-2X hybrid meta generalized gradient approximation functional, which has shown good performance in thermochemistry, thermochemical kinetics, and non-covalent interactions studies of species that do not contain metals. Single-point calculations with the 6-311++G(2d,p) basis set have been performed at the geometries optimized with the 6-31+G(d) basis set. Preliminary single-point calculations have also been performed with the smaller 6-311+G(d,p) basis set. There are no qualitative differences between the results obtained with the two basis sets, although changes of up to 6 kJ mol⁻¹ between the two set of results have been observed. As all experimental reactions were carried out in DMF at 105° C., all free energy data were calculated at this temperature using the CPCM continuum solvation model for DMF. Use of a continuum solvation model is justified as specific solute-solvent interactions (e.g., hydrogen bonds) are not expected in the studied systems. Additionally, CPCM model has shown good performance in studies of barrier heights and reaction energies of compounds in non-aqueous solutions. The free energies of reactants, transition states and products are given in FIG. 6. These values include the compression work correction associated with moving a solute from a standard-state gas-phase concentration of 1 atm to a standard-state solution-phase concentration of 1 mol L⁻¹. This effect is relevant for reactions or steps in which the molecularity between reactants and products is altered, such as in 1→2_{A}, 2_{B,C}→3_{B,C}, 4_{B}→5_{B}, 4_{C}→5_{C}, and 6_{C}→7_{C} (=5_{B}) steps, and accounts for a significant lowering of free energy changes (by ˜10.8 kJ mol⁻¹ at 105° C.). For MeOEtOTs the compression work effect leads to an increase of the energy difference between 2_{B,C} and 2_{A}, while for MeOBuOTs and MeOPenOTs it leads to a decrease of this energy difference, but such decrease is not enough to revert the energy ordering of 2_{A} and 2_{B,C}. On the other hand, in the case of MeOHexOTs such effect causes a reversion in the energies of 2_{B,C} and 2_{A} (FIG. 6). The coulombic stabilization energy due to the formation of ionic pairs, as in 3_{A}, 3_{B,C}, 4_{B} and 5_{B}, were of ˜5 kJ mol⁻¹; and such correction had little impact on the overall free energy profile.

Routes B and C are marked by the involvement or formation of oxacycloalkanes as transition states, intermediates, or products. MeOEtOTS, MeOBuOTs, MeOPenOTs, and MeOHexOTs are, thus, associated with the corresponding heterocyclic rings oxacyclopropane (oxirane, epoxide), oxacyclopentane (oxolane, tetrahydrofuran), oxacyclohexane (oxane, tetrahydropyran), and oxacycloheptane (oxepane), respectively. Whereas FIG. 4 shows that the methylation of N-pyridylporphyrins, regardless of the isomer type, is favored in the following order MeOPetOTs>MeOBuOTs>MeOHexOTs>MeOEtOTs, it is worth noting that this overall reactivity correlates roughly with the stability trend of the corresponding heterocycles tetrahydropyran (6-membered ring)>tetrahydrofuran (5-membered ring)=oxepane (7-membered ring)>>epoxide (3-membered ring),⁵¹ with the exception being the MeOHexOTs system, which shows levels of methylation similar to those of MeOEtOTs (FIG. 4); this apparent incongruity will be dealt later. For the MeOEtOTs system, in which methylation by either Routes B or C depends on the formation of unstable 3-member ring species, the direct methoxyalkylation (Route A) is considerably more favorable (by more than 39 kJ·mol⁻¹) than the other routes (compare the energy of the first transition states 2_{A} and 2_{B,C} in FIG. 6), which is consistent with the very low level of methylation verified in this case. In the MeOBuOTs and MeOPenOTs systems, the first energy barrier associated with the pre-organization of a 5- and 6-member ring is smaller in Routes B and C than that in Route A (that is, 2_{B,C}<2_{A}), although the difference in the energies of the transition states 2_{B,C} and 2_{A} (of ˜6 kJ·mol⁻¹) is significantly smaller than the corresponding energy difference for the MeOEtOTs system. However, even this relatively small difference seems to be high enough to contribute, along with other effects discussed later, with the much higher degrees of methylation (FIGS. 3 and 4) obtained in these two systems; the difference in energy is approximately equal to 2 RT, which, thus, leads to a large effect in the reaction rate constant, given its contribution to Arrhenius' equation exponential factor. Another point is the decrease in the lowest activation energy of the first step (1→2) as one goes from systems associated with the intermediacy of 3-membered to 5-membered to 6-membered ring species, i.e., MeOEtOTs to MeOBuOTs to MeOPenOTs systems, respectively. Accordingly, on changing from the MeOEtOTs to MeOBuOTs system, a decrease of ˜5 kJ·mol⁻¹ in the activation energy is predicted, while on changing from the MeOBuOTs to the MeOPenOTs system, the activation energy is decreased by a further ˜4 kJ·mol⁻¹ (FIG. 6). Another feature in the control of the reaction rates is the probability that a given atom hits the correct atom associated with the desired transformation so that a productive TS is formed. For instance, for the formation of TS 2, the N-atom of the pyridine ring must reach the C-atom directly bound to the tosylate group in MeOalkylOTs (FIG. 5), in order to yield the effective transition state 2A, at the expense of many ineffective collisions with other atoms that give rise to no reaction. Thus, the probability of such favorable encounters and effective collisions decreases as the tosylate side-chain lengthens. Therefore, it is expected that such effect should lead to a further decrease (apart from the influence of the barrier height) in likelihood of structurally organizing the system as required by the transition state 2A for the five- and six-membered ring systems, i.e., MeOBuOTs and MeOPenOTs systems, respectively, which eventually translates into a reduced effective likelihood of Route A for this longer side-chained tosylates. The difference in the magnitude of such effect between these particular MeOBuOTs and MeOPenOTs systems should be almost negligible, since they differ by just one C-atom out of 6 or 7 core atoms, respectively, in the side chains. This type of statistical effect is also expected to be relevant for the formation of the transition state 2_{B,C}, which involves an intramolecular heterocyclic ring formation (FIG. 5). The number of rotational isomers (minima) of a given chain formed by N single bonds is 3^{N,52} In the present case N coincides with the number of single bonds in the heterocyclic ring, since only these bonds are relevant for the ring-closure probability. The population of a given minimum can be connected to its energy, via Boltzmann distribution. For MeOBuOTs, the lowest energy minimum is expected to be a precursor of a particular 5-membered ring conformer corresponding to the formation of the TS 2_{B,C} out of a total of 243 possible conformers (N=5). The conformers that resemble precursors of 3- and 4-membered rings in MeOBuOTs are ineffective for yielding the desired reaction (O-atom would hit an internal, non-activated CH₂ group) and, additionally, as a consequence of ring strain,⁵¹ their energies should be considerably higher. Thus, despite the fact that they contribute with a relatively large number of conformers, their Boltzmann population are expected to be small. Therefore, it is reasonable to assume that their occurrence represents an almost negligible obstacle for the formation of TS 2_{B,C}. The same holds for MeOPenOTs, as the desired precursor for 2_{B,C} is a 6-membered ring out of 729 possible conformers (N=6) and the energies of the ineffective 5-, 4- and 3-membered ring conformers are higher and in an ascending order (5-member<4-member<3-membered ring). On the other hand, in the case of MeOHexOTs, for which there are 2187 minima (N=7), the desired reaction involves formation of a 7-membered ring, which, of all possible precursor rings, is not the lowest energy ring. Thus, formation of TS 2_{B,C} is difficult given the occurrence of ineffective 6-membered precursor ring conformer that, as a consequence of its lower energy,⁵¹ have higher Boltzmann population than the corresponding population of the desired, effective 7-membered ring conformer. Overall, the net effect in the case of MeOHexOTs is to hinder the formation of TS 2_{B,C} in comparison to what would be expected as a consequence of a sole effect of the activation barriers. These combined energetic and conformational factors explains the low level of methylation observed experimentally for this system (FIG. 4), close to that found in the MeEtOTs systems. It is suggested then that the net outcome of the statistical and energy barrier effects makes the effective formation of TS 2_{B,C} in MeOHexOTs as unfavorable as in the case of MeOEtOTs.

FIG. 6 reveals that the formation of product 3_{A} is thermodynamically more favorable than 5_{B} for both MeOEtOTs and MeOHexOTs, i.e., the methoxyalkylation route is more favorable than the methylation routes. However, whereas for MeOEtOTs the energy difference between products and reactants is ˜50 kJ·mol⁻¹, for MeOHexOTs this difference is just ˜1 kJ·mol⁻¹. Thus, for these two systems, route A (methoxyalkylation) is kinetically, as well as thermodynamically, more favorable than Routes B and C, although the thermodynamic effect is very small for MeOHexOTs. On the other hand, for MeOBuOTs and MeOPenOTs, formation of 5_{B} is thermodynamically more favorable than formation of 3_{A} by ˜23 and ˜37 kJ·mol⁻¹, respectively. Therefore, for MeOBuTs and MeOPenOTs, Routes B and C (leading to the methylation reactions) are both kinetically and thermodynamically more favorable than Route A.

For MeOEtOTs, the relative stability between the transition states 4_{B} and 4_{C} seems to be of secondary importance, since Routes B and C are avoided already in the first step, related to the formation of 2_{B,C}, as the free energy of 2_{A} is significantly lower than that of 2_{B,C} (FIG. 6). Conversely, the relative stability between the transition states 4_{B} and 4_{C} for MeOHexOTs (FIG. 6) may play a role in hindering the formation of 5_{B}, which is consistent with the low methylation yields observed experimentally (FIG. 4) Despite the fact that such difference (of ˜12.8 kJ·mol⁻¹) is relatively high, the formation of products 5_{C} is likely to be non-negligible, thus causing a further decrease in the yield of methylated pyridinium species 5_{B}. Formation of methylation products 5_{C} in relatively high amounts may be explained by the large excess of MeOalkylOTs used under experimental conditions, as the formation of TS 4_{C} involves a bimolecular reaction, whose rate depends on the concentration of tosylate. Conversely, the rate of formation of 5_{B} from TS 4_{B} is independent of MeOalkylOTs concentration. Additionally, formation of 5_{C} is also justified by the fact that MeOalkylOTs alone, heated in DMF at 105° C. has been shown experimentally to yield the methylating agent MeOTs. The prevalence of the methylation routes for MeOBuOTs and MeOPenOTs systems in comparison with MeOHexOTs may arise from a further increase of ˜2.2 kJ-mol⁻¹ in the relative stability of TS 4_{B} versus 4_{C} along with much larger relative thermodynamic stability of 3_{A} and 5_{B} (and the other aforementioned effects for MeOHexOTs).

Routes B and C develop through a common methyl oxonium salt as intermediate. The reaction of the methyl oxonium salt with its tosylate counter-ion or with the pyridine (or pyridyl moiety of N-pyridylporphyrins) represents the crucial step in defining the overall methylation as a result of Route C or Route B, respectively. The formation of each methyl oxonium cation leads to the concomitant formation of a tosylate anion in close proximity to the cation. The access of pyridine (or N-pyridylporphyrin) to the methyl oxonium cation in a timely fashion manner is not necessary granted, given that it depends on the effective diffusion of the pyridine moiety from the solution bulk to the methyl oxonium cation intermediate. Therefore, the reaction of this cation with the tosylate to yield the stable methylating agent MeOTs, which would eventually promote methylation (Route C), should occur at the expense of direct transfer of the methyl moiety from the oxonium cation intermediate to pyridine or N-pyridylporphyrin.

The compromised balance among the suggested mechanisms given by Routes A, B, and C to describe the methoxyalkylation versus methylation reactions of N-pyridylporphyrins is in agreement with experimental data depicted in FIGS. 2, 3 and 4, especially when analyzed in conjunction with the reaction times needed for full quaternization of the porphyrin isomers in various systems. In general, time needed for the completion of the methoxyalkylation reaction of N-pyridylporphyrins with 2-methoxyethyl and 6-methoxythexyl chains was similar to that observed with the corresponding alkyl analogues of equivalent chain length. The reactions with MeOBuOTs and MeOPenOTs to yield the fully quaternized MnP mixtures were remarkably slower. For example, reactions of the ortho N-pyridylporphyrin with MeOEtOTs and MeOHexOTs lasted, as anticipated, ˜24 hours, whereas full quaternization with MeOBuOTs and MeOPenOTs was achieved in a remarkably short time frame of 4 hours. These shortened reaction times are associated with higher level of methylated species, which deems pyridyl unavailable to methoxyalkylation and leads much rapidly to predominately methylated, but fully quaternized product. Effective collisions between the pyridyl group of an N-pyridylporphyrin and the activated CH₂ group of a tosylate becomes statistically less likely as the side-chain lengthens, which should result, under normal condition,³⁰ in slower reactions for longer side-chain tosylates. Conversely, methylations are considerably fast as effective collisions are more likely, given the methylating agent is available. Thus, if the methoxyalkyl tosylate is prone to rearrangement, as in MeOBuOTs and MeOPenOTs, the slow methoxyalkylation allows time for the side-chain reorganization to take place and for the in situ-generated methylating agent to accumulate to levels enough to favor methylation at the expense of methoxyalkylation. Such reaction trend results in the methylation profile given in FIGS. 3 and 4. It is worth noting that the overall picture indicates that the N-pyridylporphyrins are acting as a somewhat exotic trapping reagent and expensive sensor for the in situ formation of methylating agents in these methoxyalkyl tosylate systems.

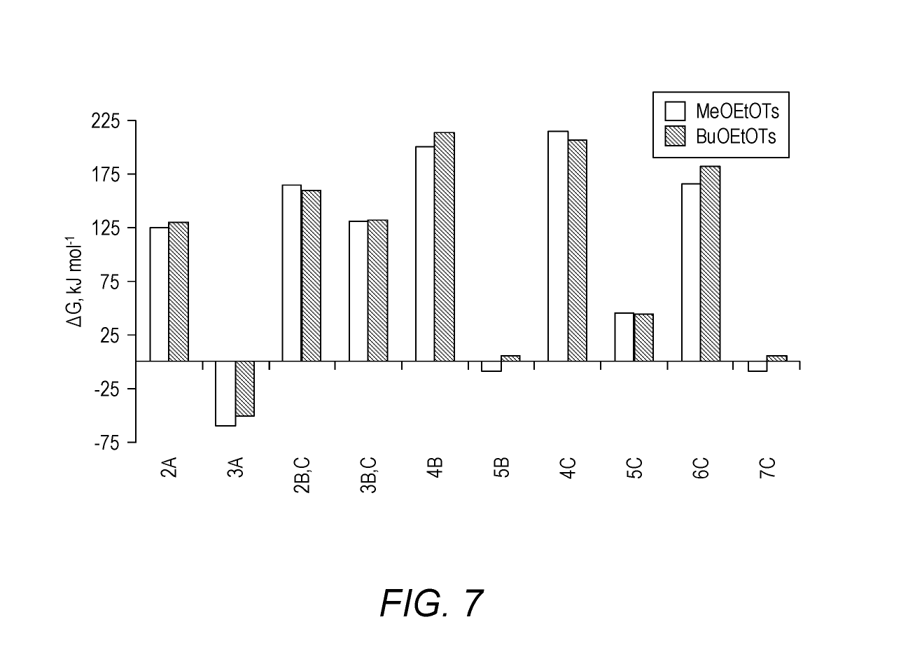

MnTnBuOE-2-PyP⁵⁺. The studies of the methoxyalkyl tosylate systems paved way for the successful synthesis of the Mn(III) 2-N-pyridyl porphyrin derivative bearing butoxyethyl side-chains, MnTnBuOE-2-PyP⁵⁺ (BMX-001) (FIG. 1). This compound is now in Phase I/II clinical Trial in the USA. The design of this compound explored the fact that the placement of the oxygen atom closer to the sulfonato group in alkoxyethyltosylate would, by analogy to the methoxyethyl tosylate system, disfavor rearrangement of the tosylate and favor, thus, alkoxyethylation versus alkylation. Indeed, the synthesis of MnTnBuOE-2-PyP⁵⁺ was accomplished⁴⁴ with no signs of competing butylation reaction. DFT calculations on competing Routes A, B, and C for the butoxyethyl tosylate system yielded energy profiles that were remarkably similar to that of the MeOEtOTs systems, except that nBuOEtOTs-based n-butylation is even slightly disfavored than MeOEtOTs-based methylation (FIG. 7). By keeping the oxygen atom 2 carbons away from the sulfonato group, formation of a 3 membered-ring as an intermediate in Routes B and C is highly disfavored, and the desired butoxyethylation reaction (Route A) is the major pathway leading to MnTnBuOE-2-PyP⁵⁺. The overall profile of MeOEtOTs and nBuOEtOTs are, thus, in excellent agreement with experimental reactivity trend.

It is worth noting that whereas MeOPenOTs and nBuOEtOTs are isomers of identical chain length, the relative position of oxygen atom within the chain places these two compounds on the very opposite sides of the reactivity trend: MeOPenOTs being extremely prone to rearrangement and favoring the corresponding methylation pathways (via Routes B and/or C), while nBuOEtOTs reacts in its own right, favoring butoxyethylation products (Route A).

Aside from the impact on the reactivity pattern of the tosylate, the relative position of the oxygen atom is also of outmost importance in controlling and defining the lipophilicity of the resulting MnP complex. The extent of solvation of the systems in which the oxygen atoms are exposed (at the end of the side-chains) relative to those buried deeply within the chains is greatly different. Whereas the methoxyhexyl derivatives are relatively hydrophilic, the butoxyethyl analogue, MnTnBuOE-2-PyP⁵⁺, is not only lipophilic but exhibit also low surfactancy character and low toxicity. With 4 cationic nitrogens, the anticipated high Mn(III)/Mn(II) reduction potential (E_{1/2}) and the high SOD-like activity were demonstrated.

Reevaluation of the purity/identity of MnTMOE-2-PyP⁵⁺ and MnTTEG-2-PyP⁵⁺ preparations. Understanding the mechanism of quaternization with oxygen-bearing p-toluenesulfonates allowed us not only to design and optimize the structure of SOD mimics, but to revisit and accurately identify the main product and by-products in the preparations of other SOD mimics and peroxynitrite scavengers reported by us, i.e., MnTMOE-2-PyP⁵⁺ and Mn PEG-ylated porphyrin (MnTTEG-2-PyP⁵⁺), and to speculate on the composition of the Fe PEG-ylated analogue, FP-15, prepared by others. While we have not tested the efficacy of MnTMOE-2-PyP⁵⁺ and MnTTEG-2-PyP⁵⁺ in vive (other than in E coli study), FP-15 has been used in different animal models. At the point we originally reported the identity and purity of our preparations of MnTMOE-2-PyP⁵⁺ and MnTTEG-2-PyP⁵⁺ we had not yet established an ESI-MS conditions which would have prevented analyte fragmentation. Thus we assigned, then,^{25, 26} the multiple peaks in mass spectra to fragmentation and losses at the ESI-MS ionization chamber, which hampered the identification of MnP contaminants in the isolated materials. With the use of heptafluorobutyric acid as an ESI-MS additive to allow ion pairing and prevented MnP fragmentation, the situation with the formerly called MnTMOE-2-PyP⁵⁺ sample was clarified in the present work: FIG. 6 indicates that the isolated preparation is, in fact, a mixture of fully quaternized MnPs in which the target MnTMOE-2-PyP⁵⁺ compound amounts to ˜70% and the remaining ˜30% relates to MnP⁵⁺ species with one or two methoxyethyl moieties being replaced by methyl groups (FIG. 6).

The revisited ESI-MS analysis of the Mn PEG-ylated compound revealed a fair number of by-products; the FP-15 which differs from MnTTEG-2-PyP⁵⁺ only by having Fe instead of Mn as the metal center, should likewise contain the analogous porphyrin-based impurities/byproducts. Due to the formation of cycles of different length during quaternization, the preparations of MnTTEG-2-PyP⁵⁺ contains not only the compound of interest, but species with different alkyl and alkoxyalkyl pyridyl substituents (Table 1). Whereas all by-products must be SOD active (given the structure-activity relationships devised for cationic porphyrins they likely have significantly different lipophilicities and, therefore, bioavailabilities, which should affect considerably their in vivo efficacy.

TABLE 1
Electrospray ionization mass spectrometry
data for MnTTEG-2-PyP⁵⁺.

| | m/z [found |
|---|---|
| MnTTEG-2-PyP⁵⁺ species | (calculated)] |
| (4PEG + HFBA⁻)⁴⁺/4 | 368.4 (368.1) |
| (1PEG/3Me + 2HFBA⁻)³⁺/3 | 430.2 (429.8) |
| (1PEG/2Me/1MeOEt + 2HFBA⁻)³⁺/3 | 445.0 (444.4) |
| (2PEG/2Me + 2HFBA⁻)³⁺/3 | 473.9 (473.8) |
| (2PEG/1Me/1MeOEt + 2HFBA⁻)³⁺/3 | 488.9 (488.5) |
| (3PEG/1Me + 2HFBA⁻)³⁺/3 | 518.0 (517.8) |
| (3PEG/1MeOEt + 2HFBA⁻)³⁺/3 | 533.0 (532.5) |
| (4PEG + 2HFBA⁻)³⁺/3 | 562.3 (561.8) |
| (4PEG + 3HFBA⁻)³⁺/2 | 949.7 (549.0) |

~1 μM solution of MnTTEG-2-PyP⁵⁺ in 1:1 v/v acetonitrile:H₂O (containing 0.01% v/v heptafluorobutyric acid (HFBA)) mixture, 20 V cone voltage.

A systematic evaluation of these ortho, meta, para Mn(III)N-pyridylporphyrins with alkoxyalkyl tosylates was undertaken and the studies indicated what type of oxygen-bearing analogues could be synthesized in purity compatible with biological demands. MnTnBuOE-2-PyP⁵⁺ emerged and is presently entering Clinical Trials as radioprotector.

The studies reported herein demonstrate that N-methylated pyridyl species in N-methoxyalkylpyridylporphyrins originate from unanticipated rearrangement mechanisms rather than from impurities in p-toluenesulfonate, solvent or starting non-alkylated porphyrin. The possibility of preparing reasonably pure (>95%) meta N-pyridylporphyrins fully quaternized with 4-methoxybutyl and 5-methoxypentyl substituents was abandoned, as well as the synthesis of ortho and para N-methoxyalkylpyridylporphyrins. The studies on the mechanism of N-alkoxyalkyl derivatization of porphyrin pyridyls led to the synthesis of MnTnBuOE-2-PyP⁵⁺ and prove that the instability associated with the formation of small 3-membered ring minimized the likelihood of butoxyethyl tosylate side-chain rearrangement and consequent formation of undesired by-products. The compound retains the powerful redox properties of analogous ortho MnP⁵⁺ and, as anticipated, is 4-5-fold less toxic due to the oxygen atoms disrupting micellar properties of analogous alkyl chains. The previous success on the synthesis of hexoxyethyl analogue, MnTnHexOE-2-PyP⁵⁺, is also explained by the unfavorable three-membered ring formation during quaternization leading the isolation of a compound of as high purity as that of MnTnBuOE-2-PyP⁵⁺. The knowledge obtained herein is invaluable to the synthesis of N-alkoxyalkylpyridylporphyrins with oxygen-atom as close to the pyridyl groups as possible to minimize competing tosylate rearrangements and the formation of unwanted species that may lead to undesired alkylated products. Thus, the work described herein led to both the design of MnTnBuOE-2-PyP⁵⁺ and the understanding on why this compound is actually amenable to preparation in high yield/purity whereas other related ones give rise to non-prospective mixtures as redox-active therapeutics.

### Example 10

### Synthesis of H₂TnHexOE-2-PyP⁴⁺ and MnTnHexOE-2-PyP⁵⁺

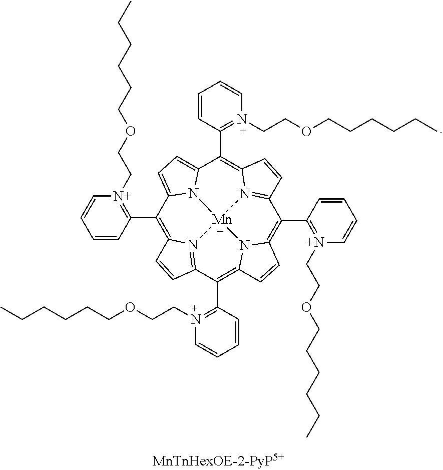

### H₂TnHexOE-2-PyPC₄, meso-tetrakis(N-(2′-n-hexoxyethyl)pyridinium-2-yl)porphyrin tetrachloride

H₂T-2-PyP (70 mg, 0.113 mmol) was dissolved in 4 mL of DMF, preheated for ˜5 min at 115° C., and the 8.5 g of 2-n-hexoxyethyl p-toluenesulfonate (0.028 mol) was added. The course of N-quaternization was followed by thin-layer chromatography (TLC) on silica gel plates using acetonitrile:KNO₃₍ₛₐₜ₎:water=8:1:1 as a mobile phase. Also, methanol/chloroform (¼) solvent system was used to monitor the reaction progress. The reaction was completed within 48 hours. Porphyrin was precipitated from the reaction mixture by diethyl ether, filtered and washed with diethyl ether (5×30 mL). The porphyrin tosylate was then dissolved in 100 mL of hot water and precipitated as the PF₆⁻ salt with saturated aqueous solution of NH₄PF₆. The precipitate was thoroughly washed with diethyl ether. The dried precipitate was then dissolved in acetone, solution filtered and porphyrin precipitated from it as a chloride salt with saturated acetone solution of methyl-tri-n-octylammonium chloride. The precipitate was washed with acetone and dissolved in water. The double precipitation was repeated once again to assure the highest purity of preparation. The porphyrin was dried in vacuum oven in the form of Cl⁻ salt. Elemental analysis: H₂TnHexOE-2-PyPCl₄-8H₂O: Anal. Calcd for C₇₂H₁₁₀Cl₄N₈O₁₂: H, 7.8; C, 60.84; N, 7.88%. Found: H, 7.72; C, 60.56; N, 7.92%. 264.4 (4.38), 419.4 (5.35), 513.5 (4.27), 545.5 (3.64), 586.4 (3.86), 640 (3.43). Electrospray ionization mass spectrometry (ESI-MS) data, species [m/z, found (calculated)]: [H₂P⁴⁺+HFBA⁻]³⁺/3 [449.4 (449.2)], [H₂P⁴⁺+2HFBA⁻]²⁺/2 [780.2 (780.4)], [H₂P]⁴⁺/4283.6 (283.7) [H₂P⁴⁺−H⁺]³⁺/3378.1 (377.9) [H₂P⁴⁺− H⁺+HFBA⁻]²⁺/2 673.3 (673.4), [H₂P⁴⁺+H⁺+3HFBA⁻]²⁺/2 887.0 (887.3). TLC retention factor, R_{f} (silica gel plates using acetonitrile:KNO₃₍ₛₐₜ₎:water=8:1:1 as a mobile phase) 0.53.

### MnTnHexOE-2-PyPCl₅, Mn(III) meso-tetrakis(N-(2′-n-hexoxyethyl)pyridinium-2-yl)porphyrin pentachloride

The pH of 80 mL of H₂TnHexOE-2-PyPCl₄ aqueous solution (100 mg, 0.078 mmol) was adjusted to 10.9 and a 20-fold excess of MnCl₂ (310 mg, 1.55 mmol) was added into the solution while stirring at 25° C. for 2.5 hours until metalation was completed. The course of metalation was followed on silica gel TLC plates using acetonitrile:KNO₃₍ₛₐₜ₎:water=8:1:1 as a mobile phase. The pH of the solution was periodically adjusted to 7.2. Additionally, the course of metalation was monitored as a disappearance of porphyrin ligand fluorescence under uv light at ˜350 nm. The porphyrin solution was filtered first through a coarse, then through fine filter paper. The Mn porphyrin was precipitated as a PF₆ salt with saturated aqueous solution of NH₄PF₆. The precipitate was thoroughly washed with diethyl ether. The dried precipitate was then dissolved in acetone, filtered and precipitated as the chloride salt with saturated acetone solution of methyl-tri-n-octylammonium chloride. The precipitate was washed with acetone and dissolved in water. The double precipitation was repeated once again to assure the highest purity of porphyrin and complete removal of free manganese species. Elemental analysis: MnTnHexOE-2-PyPCl₅.8.5H₂O: Anal. Calcd for C₆₄H₉₄Cl₅MnN₈O₉: H, 7.23; C, 56.93; N, 7.38; Cl, 11.67%. Found: H, 7.05; C, 56.58; N, 7.68; Cl, 11.28%. UV-visible, m nm (log c): 212.5 (4.72), 261.7 (4.56), 365.4 (4.74), 411.4 (4.39), 455.5 (5.26), 561.1 (4.16), 786.5 (3.38). Electrospray ionization mass spectrometry (ESI-MS) data, species [m/z, found (calculated)]: [MnP⁵⁺+HFBA⁻]⁴⁺/4 [350.2 (350.2)], [MnP⁵⁺+2HFBA⁻]³⁺/3 [537.7 (537.9)], [MnP⁴⁺+3HFBA⁻]²⁺/2 [913.0 (913.3)]. TLC retention factor, R_{f} (silica gel plates using acetonitrile:KNO₃₍ₛₐₜ₎:water=8:1:1 as a mobile phase) 0.50.

### Comparative Example

### Prior Art Synthesis of BMX-001

Z. Rajic et al., Free Radical Biology & Medicine 53, 1828-1834 (2012) describes a prior synthesis of MnTnBuOE-2-PyP⁵⁺ at page 1830, first column therein.

The inventors of the present invention have discovered that the procedures reported in Z. Rajic et al. do not provide a composition of a compound of Formula I as described herein in which the amount of contaminants, such as free manganese and/or alternate forms of pyridyl porphyrins, are substantially limited or controlled. For example, it has now been found that the pH range during metalation should be closely controlled to avoid generating alternate forms of pyridyl porphyrins, as described above. Further, the procedure described in Z. Rajic et al. does not recognize the need to optimize the volumes of solvent (DMF), equivalents of BMX-001-1, and equivalents of Oct₃N to maximize conversion of H₂T-2-PyP to BMX-001-2 and to minimize the formation of impurities during prolonged heating at 105° C. Further, the prior procedure does not recognize the need to include a flocculant in isolation of the BMX-001-2-OTs by precipitation, extraction of desired product from flocculant with water and direct conversion to BMX-001-2-PF₆, which helps to avoid problematic aqueous workup where the product partitions into both aqueous and organic phases, particularly when larger quantities thereof are being produced. Further, the procedure described in Z. Rajic et al. does not recognize the importance of impurity profile in initial materials, i.e. in 2-butoxyethanol, and in intermediate products, i.e. in BMX-001-1, which may lead to the formation of extremely reactive reagents, that increase the impurity levels in subsequent steps.

The foregoing is illustrative of the present invention, and is not to be construed as limiting thereof. The invention is defined by the following claims, with equivalents of the claims to be included therein.

That which is claimed is:

1. A method of making a compound of Formula 001: 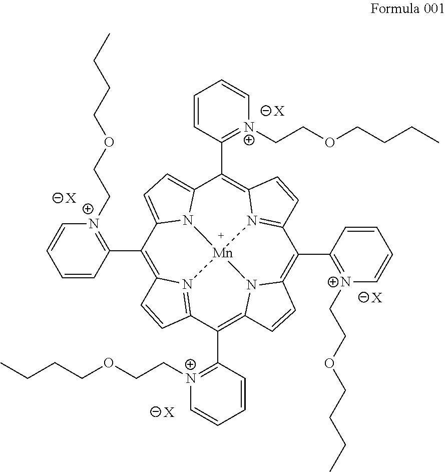
wherein X is an anion (e.g., Cl, PF₆, tosylate, besylate, mesylate, etc.), the method comprising:
(a) providing a compound of Formula 001-2: 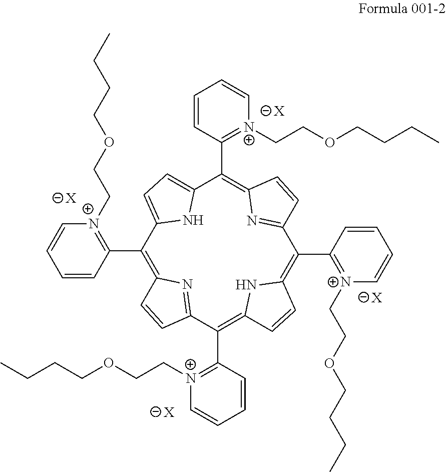
in an aqueous solution at a pH of from 10 to 12 (e.g., 11), then
(b) combining MnCl₂×4 H₂O into said aqueous solution to produce a mixed solution; and then
(c) oxygenating said mixed solution while
(d) monitoring and periodically adjusting the pH thereof to maintain a pH thereof between 7.6 or 7.8 and 8.2 or 8.4 (e.g., to maintain a pH of 8), while continuing oxygenating of said mixed solution for a time sufficient to produce said compound of Formula 001.

2. The method of claim 1, wherein said monitoring step is carried out with a pH sensor or detector contacting said mixed solution during said oxygenating step.

3. The method of claim 1 or 2, wherein said periodically adjusting step is carried out by adding a base to said mixed solution when said monitored pH is less than 7.6 or 7.8, and/or adding an acid to said mixed solution when said monitored pH is greater than 8.2 or 8.4.

4. The method of claim 1 to 3, wherein said step of providing said compound of Formula 001-2 is carried out by providing a composition of pyridyl porphyrins,
said composition comprising said compound of Formula 001-2 in a combination with other different pyridyl porphyrins,
wherein at least 80, 85, 90, or 95 percent by weight of all pyridyl porphyrins in said composition is said compound of Formula 001-2.

5. The method of claim 1 to 4, wherein at least 80, 85, 90, or 95 percent by weight of all manganese pyridyl-porphyrins produced from said compound of Formula 001-2, or said composition comprising said compound of Formula 001-2, is said compound of Formula 001.

6. The method of claim 4 or claim 5 dependent on claim 4, wherein not more than 20, 15, 10 or 5 percent by weight of all manganese pyridyl-porphyrins produced from said method consist of compounds of Formulas (iii), (iv), (v), (vi), (vii) and (viii): 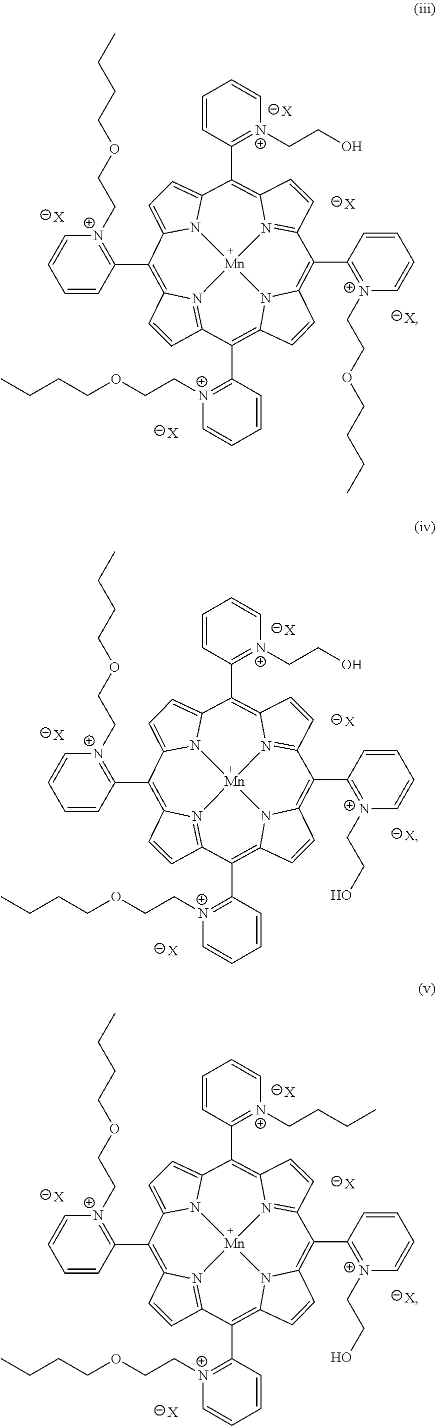 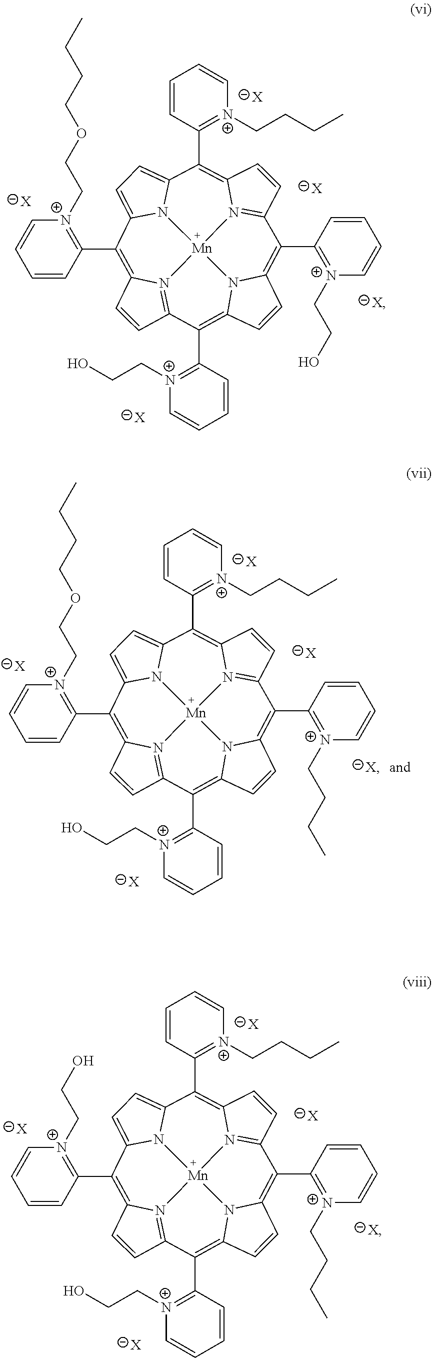
wherein X is an anion as given above.

7. A method of making a compound of Formula 001-2 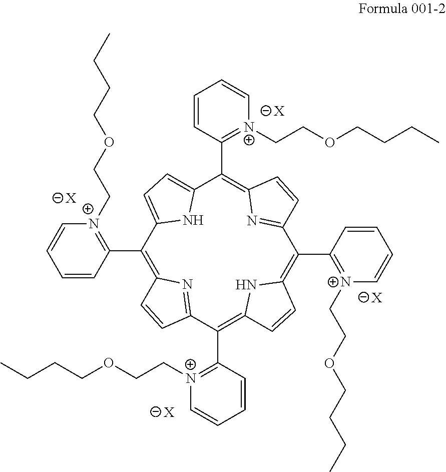
wherein X is an anion (e.g., CI, PF₆), the method comprising the steps of:
(a) providing compound H₂T-2-PyP in a heated solution of a polar aprotic solvent (e.g., dimethylformamide) with tri-n-octylamine (Oct₃N) 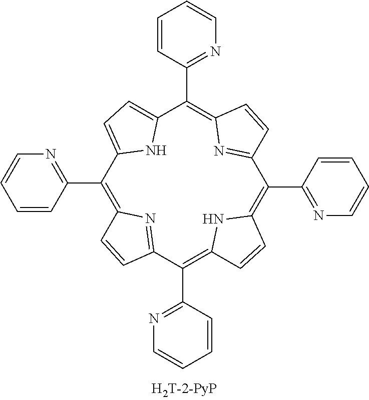 [SMILES: C1=Cc2nc1c(-c1ccccn1)c1ccc([nH]1)c(-c1ccccn1)c1nc(c(-c3ccccn3)c3ccc([nH]3)c2-c2ccccn2)C=C1]
wherein said heated solution is purged of oxygen (e.g., by sparging with an inert gas such as nitrogen or argon); then
(b) combining said heated solution with 2-butoxyethyl p-toluenesulfonate to produce a liquid mixture;
(c) maintaining said liquid mixture at an elevated temperature (e.g., 85 to 105° C.) for a time (e.g., 45-60 hours) sufficient to produce an intermediate product (i.e., BMX-001-2-OTs) in an intermediate liquid; then
(d) optionally combining said intermediate liquid with a flocculant (e.g. an organic or inorganic flocculant, such as powdered cellulose (e.g., Solka floe)) so that the intermediate product partitions with the flocculant:
(e) separating said flocculant when present from said intermediate liquid (e.g., by filtration, settling, centrifugation, or a combination thereof), then
(f) washing said flocculant with an aqueous wash solution to produce an aqueous solution carrying said intermediate reaction product; and
(g) combining said aqueous solution with a salt of said anion to produce said compound of Formula 001-2.

8. The method of claim 7, wherein said combining step (b) is carried out with a 2-butoxyethyl p-toluenesulfonate composition comprising less than 1 weight percent (relative to said 2-butoxyethyl p-toluenesulfonate) of tetrahydrofuran (THF).

9. The method of claim 7 or 8, wherein said tri-n-octylamine is included in an amount in a range of about 5 to about 25 molar excess over H₂T-2-PyP.

10. A pharmaceutical composition comprising metallated pyridyl-porphyrins in a pharmaceutically acceptable carrier, wherein at least 80, 85, 90 or 95 percent by weight of all of said metallated pyridyl-porphyrins in said composition is a compound of Formula 001: 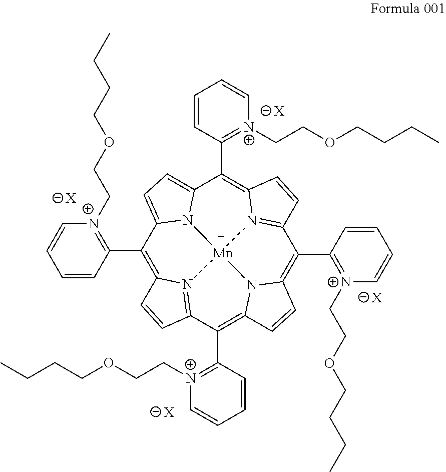
wherein X is a pharmaceutically acceptable anion.

11. The composition of claim 10, wherein X is selected from the group consisting of Cl, PF₆ tosylate, mesylate, and besylate.

12. The composition of claim 10 or 11, wherein said carrier is an aqueous carrier.

13. The composition of claim 10 to 12, wherein said composition comprises, excluding the weight of said carrier, less than 1, 1.3 or 2 percent by weight free manganese.

14. The composition of claims 10 to 13, wherein not more than 20, 15, 10 or 5 percent by weight of all metallated pyridyl-porphyrins in said composition consist of compounds of Formulas (iii), (iv), (v), (vi), (vii) and (viii): 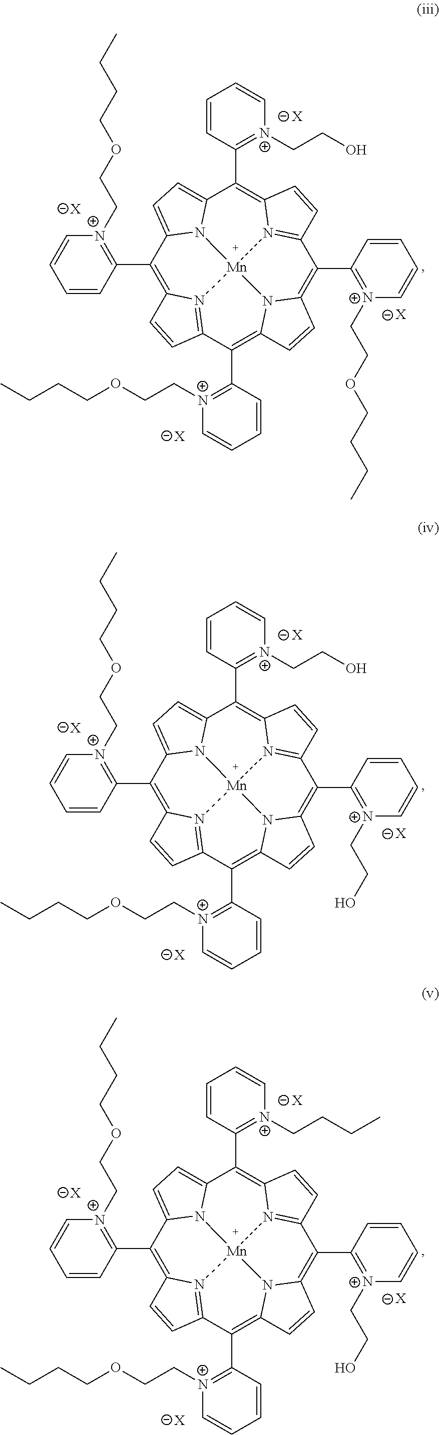 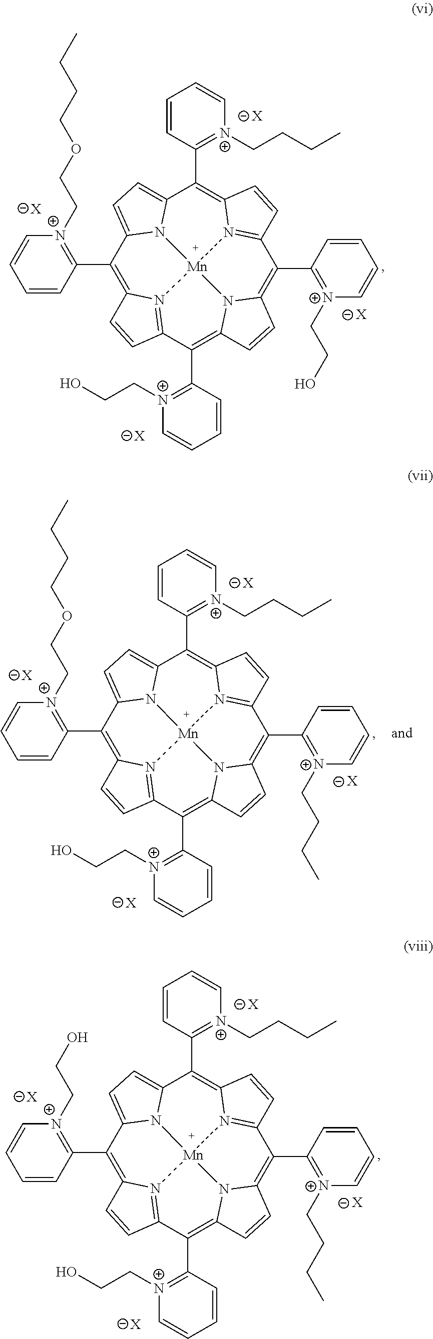
where X is an anion as given above.

15. The composition of claim 10 to 14 for use in treating inflammatory lung disease, neurodegenerative disease, radiation injury, cancer, diabetes, cardiac conditions, or sickle cell disease.

16. A method of making a compound of Formula 002: 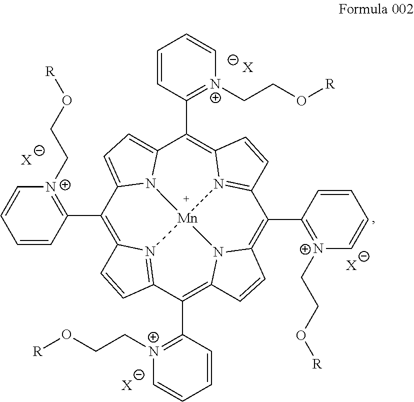
wherein each R is independently a C4-C12 alkyl and X is an anion (e.g., Cl, PF₆, tosylate, besylate, mesylate, etc.), the method comprising:
(a) providing a compound of Formula 002-2: 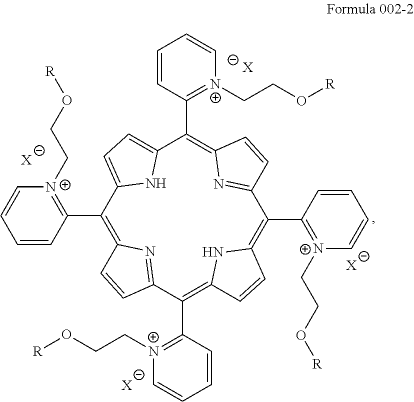
wherein each R is independently a C4-C12 alkyl and X is an anion (e.g., Cl, PF₆, tosylate, besylate, mesylate, etc.), in an aqueous solution at a pH of from 10 to 12 (e.g., 11), then
(b) combining MnCl₂×4 H₂O into said aqueous solution to produce a mixed solution; and then
(c) oxygenating said mixed solution while
(d) monitoring and periodically adjusting the pH thereof to maintain a pH thereof between 7.6 or 7.8 and 8.2 or 8.4 (e.g., to maintain a pH of 8), while continuing oxygenating of said mixed solution for a time sufficient to produce said compound of Formula 002.

17. The method of claim 16, wherein said monitoring step is carried out with a pH sensor or detector contacting said mixed solution during said oxygenating step.

18. The method of claim 16 or 17, wherein said periodically adjusting step is carried out by adding a base to said mixed solution when said monitored pH is less than 7.6 or 7.8, and/or adding an acid to said mixed solution when said monitored pH is greater than 8.2 or 8.4.

19. The method of claim 16 to 18, wherein said step of providing said compound of Formula 002-2 is carried out by providing a composition of pyridyl porphyrins,
said composition comprising said compound of Formula 002-2 in a combination with other different pyridyl porphyrins,
wherein at least 80, 85, 90, or 95 percent by weight of all pyridyl porphyrins in said composition is said compound of Formula 002-2.

20. The method of claim 16 to 19, wherein at least 80, 85, 90, or 95 percent by weight of all manganese pyridyl-porphyrins produced from said compound of Formula 002-2, or said composition comprising said compound of Formula 002-2, is said compound of Formula 002.

21. The method of claim 19 or claim 20 dependent on claim 19, wherein not more than 20, 15, 10 or 5 percent by weight of all manganese pyridyl-porphyrins produced from said method consist of compounds of Formulas (iiia), (iva), (va), (via), (viia) and (viiia): 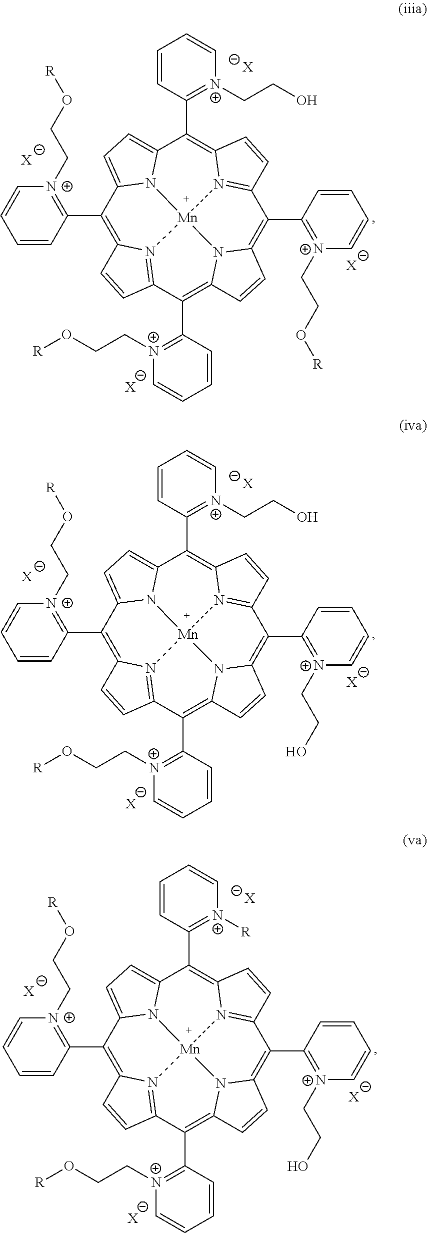 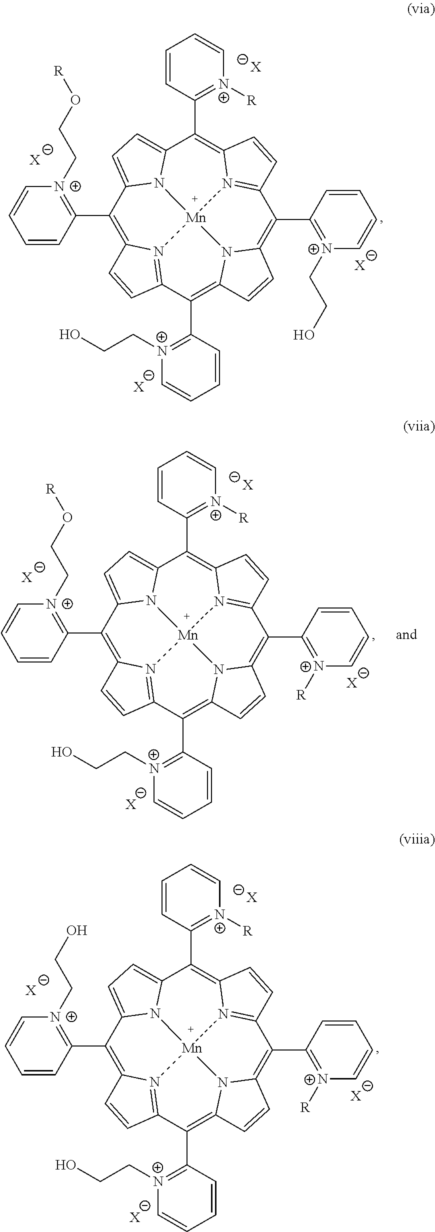
wherein each R is independently a C4-C12 alkyl and X is an anion as given above.

22. A method of making a compound of Formula 002-2 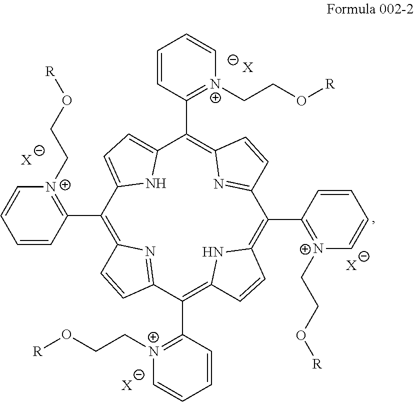
wherein each R is independently a C4-C12 alkyl and X is an anion (e.g., Cl, PF₆, tosylate, besylate, mesylate, etc.), the method comprising the steps of:
(a) providing compound H₂T-2-PyP in a heated solution of a polar aprotic solvent (e.g., dimethylformamide) with tri-n-octylamine (Oct₃N) 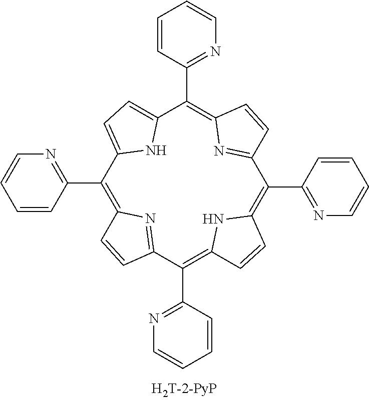 [SMILES: C1=Cc2nc1c(-c1ccccn1)c1ccc([nH]1)c(-c1ccccn1)c1nc(c(-c3ccccn3)c3ccc([nH]3)c2-c2ccccn2)C=C1]
wherein said heated solution is purged of oxygen (e.g., by sparging with an inert gas such as nitrogen or argon); then
(b) combining said heated solution with 2-alkoxyethyl p-toluenesulfonate to produce a liquid mixture;
(c) maintaining said liquid mixture at an elevated temperature (e.g., 85 to 105° C.) for a time (e.g., 45-60 hours) sufficient to produce an intermediate product (i.e., BMX-001-2-OTs) in an intermediate liquid; then
(d) optionally combining said intermediate liquid with a flocculant (e.g. an organic or inorganic flocculant, such as powdered cellulose (e.g., Solka floe)) so that the intermediate product partitions with the flocculant;
(e) separating said flocculant when present from said intermediate liquid (e.g., by filtration, settling, centrifugation, or a combination thereof), then
(f) washing said flocculant with an aqueous wash solution to produce an aqueous solution carrying said intermediate reaction product; and
(g) combining said aqueous solution with a salt of said anion to produce said compound of Formula 002-2.

23. The method of claim 22, wherein said combining step (b) is carried out with a 2-alkoxyethyl p-toluenesulfonate composition comprising less than 1 weight percent (relative to said 2-alkoxyethyl p-toluenesulfonate) of tetrahydrofuran (THF).

24. The method of claim 22 or 23, wherein said tri-n-octylamine is included in an amount in a range of about 5 to about 25 molar excess over H₂T-2-PyP.

25. A pharmaceutical composition comprising metallated pyridyl-porphyrins in a pharmaceutically acceptable carrier, wherein at least 80, 85, 90 or 95 percent by weight of all of said metallated pyridyl-porphyrins in said composition is a compound of Formula 002: 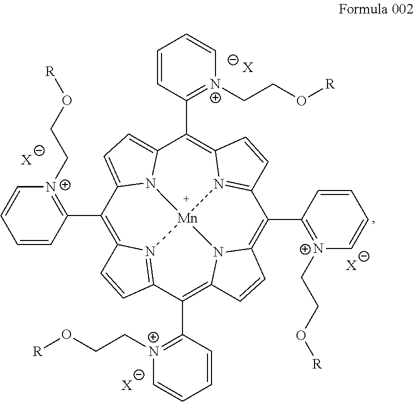
wherein each R is independently a C4-C12 alkyl and X is a pharmaceutically acceptable anion.

26. The composition of claim 25, wherein X is selected from the group consisting of Cl, PF₆ tosylate, mesylate, and besylate.

27. The composition of claim 25, wherein R in a compound of Formula 002 is a C4 alkyl, a C5 alkyl or a C6 alkyl.

28. The composition of claim 25 to 27, wherein said carrier is an aqueous carrier.

29. The composition of claim 25 to 28, wherein said composition comprises, excluding the weight of said carrier, less than 1, 1.3 or 2 percent by weight free manganese.

30. The composition of claims 25 to 29, wherein not more than 20, 15, 10 or 5 percent by weight of all metallated pyridyl-porphyrins in said composition consist of compounds of Formulas (iiia), (iva), (va), (via), (viia) and (viia): 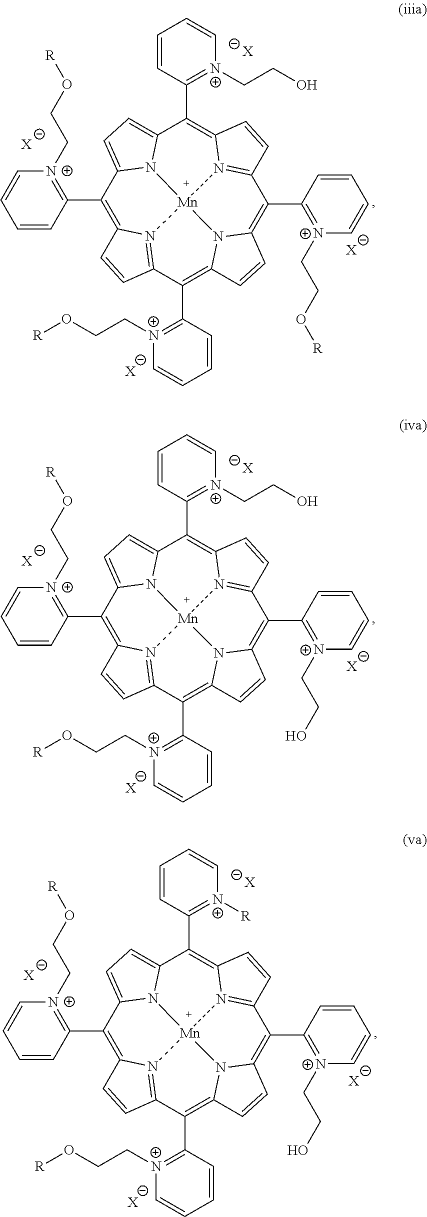 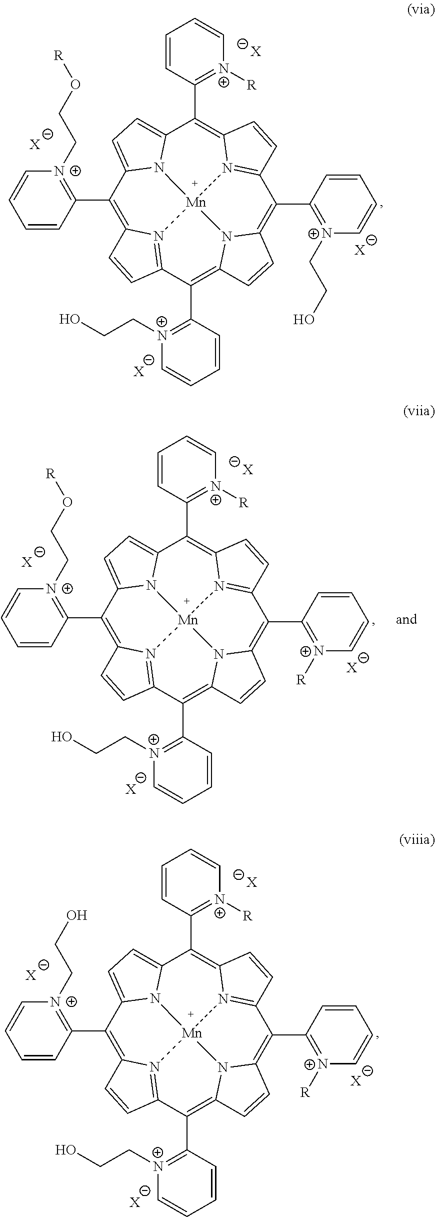
wherein each R is independently a C4-C12 alkyl and X is an anion as given above.

31. The composition of claim 25 to 30 for use in treating inflammatory lung disease, neurodegenerative disease, radiation injury, cancer, diabetes, cardiac conditions, or sickle cell disease.
